(19) United States
(12) Patent Application Publication
(10) Pub. No.: US 2023/0085724 A1
(43) Pub. Date: Mar. 23, 2023

(54) METHODS AND COMPOSITIONS FOR TREATING CANCER WITH IMMUNE CELLS

(71) Applicant: NeoTX Therapeutics Ltd., Rehovot (IL)

(72) Inventors: Michal Shahar, Rishon LeZion (IL); Asher Nathan, Rishon LeZion (IL); Yael Sagi, Rehovot (IL)

(21) Appl. No.: 17/909,212

(22) PCT Filed: Mar. 5, 2021
(86) PCT No.: PCT/IB2021/000934

§ 371 Date: Sep. 2, 2022

Related U.S. Application Data
(60) Provisional application No. 62/985,553, filed on Mar. 5, 2020.

(51) Int. Cl. A61K 35/17; A61K 45/06; A61P 35/00

(52) U.S. Cl. CPC A61K 35/17; A61K 45/06; A61P 35/00

(57) ABSTRACT

The invention provides methods or compositions for treating cancer using an immune cell, e.g, a T-cell, e.g., a CAR T-cell, optionally in combination with a superantigen conjugate. The invention also provides methods for making immune cells, e.g, T-cells, e.g, CAR T-cells, for use in the treatment of cancer.

### CROSS REFERENCE TO RELATED APPLICATIONS

This application claims the benefit of, and priority to, U.S. Provisional Patent Application Ser. No. 62/985,553, filed Mar. 5, 2020, the entire contents of which are hereby incorporated by reference in their entirety.

### FIELD OF THE INVENTION

The invention relates generally to compositions and methods for treating cancer in a subject, and, more particularly, the invention relates to methods and compositions for treating cancer using an immune cell optionally in combination with a superantigen conjugate, and methods of making immune cells for use in the treatment of cancer.

### BACKGROUND

According to the American Cancer Society, more than one million people in the United States are diagnosed with cancer each year. Cancer is a disease that results from uncontrolled proliferation of cells that were once subject to natural control mechanisms but have been transformed into cancerous cells that continue to proliferate in an uncontrolled manner.

Chimeric antigen receptors (CARs) are synthetic receptors that retarget immune cells, e.g., T cells, to tumor surface antigens (Sadelain et al. (2003), NAT. REV. CANCER. 3(1):35-45, Sadelain et al. (2013) CANCER DISCOVERY 3(4):388-398). CARs provide both antigen binding and immune cell activation functions. Initially, CARs contained an antibody-based tumor-binding element, such as a single chain Fv (scFv), that is responsible for antigen recognition linked to either CD3zeta or Fc receptor signaling domains, which trigger T-cell activation. Later CAR constructs included additional activating and costimulatory signaling domains which have led to encouraging results in patients with chemorefractory B-cell malignancies (Brentjens et al. (2013) SCI. TRANS. MED. 5(177): 177ra38, Brentjens et al. (2011) BLOOD 118(18): 4817-4828, Davila et al. (2014) SCI. TRANS. MED. 6(224): 224ra25, Grupp et al. (2013) N. ENGL. J. MED. 368(16): 1509-1518, Kalos et al. (2011) SCI. TRANS. MED. 3(95): 95ra73). CAR therapies have been approved from the treatment of subsets of patients with relapsed or refractory large B cell lymphoma and subsets of patients with acute lymphoblastic leukemia (ALL). However, CAR therapies targeting solid tumors have proven more challenging (See, for example, Martinez et al. (2019) FRONT IMMUNOL 10:128).

Despite the significant advances being made in cancer treatment and management, there is still an ongoing need for new and effective therapies for treating and managing cancer.

### SUMMARY OF THE INVENTION

The invention is based, in part, upon the discovery that a targeted immune response against a cancer in a subject can be enhanced by combining a superantigen conjugate comprising a superantigen (e.g., engineered Staphylococcal enterotoxin superantigen SEA/E-120) covalently linked to a targeting moiety that binds a cancer antigen with an immune cell (e.g., a T-cell, e.g., a chimeric antigen receptor (CAR) T-cell). Furthermore, it has been discovered that an anti-cancer treatment using a superantigen conjugate and immune cell can be enhanced by using immune cells that express T-cell receptors that bind to the superantigen (e.g., T-cell receptors comprising T-cell receptor β variable 7-9 (TRBV7-9)).

Accordingly, in one aspect, the invention provides a method of treating cancer in a subject in need thereof. The method comprises administering to the subject: (i) an effective amount of a superantigen conjugate comprising a superantigen covalently linked to a targeting moiety that binds a first cancer antigen expressed by cancerous cells within the subject; and (ii) an effective amount of an immune cell (e.g., an isolated immune cell) comprising an exogenous nucleotide sequence encoding a chimeric antigen receptor (CAR) that binds a second cancer antigen expressed by cancerous cells within the subject.

In certain embodiments, the superantigen comprises Staphylococcal enterotoxin A or an immunologically reactive variant and/or fragment thereof. In certain embodiments, the superantigen comprises the amino acid sequence of SEQ ID NO: 3, or an immunologically reactive variant and/or fragment thereof.

In certain embodiments, the targeting moiety is an antibody. In certain embodiments, the antibody is an anti-5T4 antibody, for example, anti-5T4 antibody comprising a Fab fragment that binds a 5T4 cancer antigen. In certain embodiments, the anti-5T4 antibody comprises a heavy chain comprising amino acid residues 1-458 of SEQ ID NO: 8 and a light chain comprising amino acid residues 1-214 of SEQ ID NO: 9.

In certain embodiments, the superantigen conjugate comprises a first protein chain comprising SEQ ID NO: 8 and a second protein chain comprising SEQ ID NO: 9.

In certain embodiments, the immune cell (e.g., the isolated immune cell) is selected from a T-cell, a natural killer cell (NK), and a natural killer T-cell (NKT). In certain embodiments, the immune cell (e.g., the isolated immune cell) is a T-cell, for example, a T-cell comprising a T-cell receptor comprising TRBV7-9.

In certain embodiments, the first and second cancer antigen are the same. In certain embodiments, the first and second cancer antigen are different. In certain embodiments, the first and/or second cancer antigen is selected from 5T4, mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD47, CD49f, CD56, CD74, CD123, CD133, CD138, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-a and β (FRa and β), Ganglioside G2 (GD2), Ganglioside G3 (GD3), an Epidermal Growth Factor Receptor (EGFR), Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Ra2), K-light chain, kinase insert domain receptor (KDR), Lewis A (CA19.9), Lewis Y (LeY), LI cell adhesion molecule (LICAM), melanoma-associated antigen 1 (melanoma antigen family A1, MAGE-A1), Mucin 16 (MUC-16), Mucin 1 (MUC-1), KG2D ligands, cancer-testis antigen NY-ESO-1, tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-IH, HERK-V, IL-1 IRa, Latent Membrane Protein 1 (LMP1), Neural cell-adhesion molecule (N-CAM/CD56), programmed cell death receptor ligand 1 (PD-L1), B Cell Maturation Antigen (BCMA), and Trail Receptor (TRAIL R). In certain embodiments, the first and/or second cancer antigen is selected from 5T4, EpCAM, HER2, EGFRViii, and IL13Rα2, for example, the first cancer antigen is 5T4.

In certain embodiments, the superantigen conjugate and the immune cell (e.g., the isolated immune cell) are administered separately. In certain embodiments, the superantigen conjugate and the immune cell (e.g., the isolated immune cell) are administered in combination. In certain embodiments, the superantigen conjugate and the immune cell (e.g., the isolated immune cell) are administered at the same time. In certain embodiments, the superantigen conjugate and the immune cell (e.g., the isolated immune cell) are administered at different times.

In certain embodiments, the method further comprises administering to the subject a PD-1 based inhibitor, for example, a PD-1 or PD-L1 inhibitor. In certain embodiments, the PD-1 inhibitor is an anti-PD-1 antibody, e.g., an anti-PD-1 antibody selected from nivolumab pembrolizumab, and cemiplimab. In certain embodiments, the PD-L1 inhibitor is an anti-PD-L1 antibody, e.g., an anti-PD-L1 antibody selected from atezolizumab, avelumab, and durvalumab.

In another aspect, the invention provides a pharmaceutical composition comprising: (i) a superantigen conjugate comprising a superantigen covalently linked to a targeting moiety that binds a first cancer antigen expressed by cancerous cells within the subject; (ii) an immune cell (e.g., an isolated immune cell) comprising an exogenous nucleotide sequence encoding a chimeric antigen receptor (CAR) that binds a second cancer antigen expressed by cancerous cells within the subject; and (iii) a pharmaceutically acceptable carrier or diluent. In another aspect, the invention provides a method of treating cancer in a subject in need thereof. The method comprises administering to the subject an effective amount of the foregoing pharmaceutical composition.

In another aspect, the invention provides a method of expanding T-cells (e.g., isolated T-cells) comprising a T-cell receptor comprising TRBV7-9. The method comprises contacting the T-cells with (i) a superantigen comprising Staphylococcal enterotoxin A or an immunologically reactive variant and/or fragment thereof, and/or (ii) a cell comprising a major histocompatibility complex (MHC) class II. In certain embodiments, the superantigen comprises the amino acid sequence of SEQ ID NO: 3, or an immunologically reactive variant and/or fragment thereof. In certain embodiments, the cell comprising an MHC class II is an antigen presenting cell (APC). In certain embodiments, the cell comprising an MHC class II is a monocyte and/or a B-cell.

In another aspect, the invention provides a method of producing a T-cell (e.g., an isolated T-cell) for use in the treatment of a subject. The method comprises contacting T-cells (e.g., T-cells isolated from the subject) with (i) a superantigen comprising Staphylococcal enterotoxin A or an immunologically reactive variant and/or fragment thereof, and/or (ii) a cell comprising a major histocompatibility complex (MHC) class II. In certain embodiments, the superantigen comprises the amino acid sequence of SEQ ID NO: 3, or an immunologically reactive variant and/or fragment thereof. In certain embodiments, the cell comprising an MHC class II is an antigen presenting cell (APC). In certain embodiments, the cell comprising an MHC class II is a monocyte and/or a B-cell.

In another aspect, the invention provides a method of producing a chimeric antigen receptor (CAR) T-cell. The method comprises: (a) contacting T-cells (e.g., T-cells isolated from a subject) with (i) a superantigen comprising Staphylococcal enterotoxin A or an immunologically reactive variant and/or fragment thereof, and/or (ii) a cell comprising a major histocompatibility complex (MHC) class II; and (b) modifying the T-cells to comprise an exogenous nucleotide sequence encoding a chimeric antigen receptor (CAR). In certain embodiments, the superantigen comprises the amino acid sequence of SEQ ID NO: 3, or an immunologically reactive variant and/or fragment thereof. In certain embodiments, the cell comprising an MHC class II is an antigen presenting cell (APC). In certain embodiments, the cell comprising an MHC class II is a monocyte and/or a B-cell.

In another aspect, the invention provides a method of producing a chimeric antigen receptor (CAR) T-cell. The method comprises: (a) modifying T-cells (e.g., T-cells isolated from a subject) to comprise an exogenous nucleotide sequence encoding a chimeric antigen receptor (CAR); and (b) contacting the T-cells with (i) a superantigen comprising Staphylococcal enterotoxin A or an immunologically reactive variant and/or fragment thereof, and/or (ii) a cell comprising a major histocompatibility complex (MHC) class II. In certain embodiments, the superantigen comprises the amino acid sequence of SEQ ID NO: 3, or an immunologically reactive variant and/or fragment thereof. In certain embodiments, the cell comprising an MHC class II is an antigen presenting cell (APC). In certain embodiments, the cell comprising an MHC class II is a monocyte and/or a B-cell.

In another aspect, the invention provides a method of producing a chimeric antigen receptor (CAR) T-cell. The method comprises modifying T-cells (e.g., isolated T-cells) to comprise an exogenous nucleotide sequence encoding a chimeric antigen receptor (CAR), wherein the T-cells have been contacted with (i) a superantigen comprising Staphylococcal enterotoxin A or an immunologically reactive variant and/or fragment thereof, and/or (ii) a cell comprising a major histocompatibility complex (MHC) class II. In certain embodiments, the superantigen comprises the amino acid sequence of SEQ ID NO: 3, or an immunologically reactive variant and/or fragment thereof. In certain embodiments, the cell comprising an MHC class II is an antigen presenting cell (APC). In certain embodiments, the cell comprising an MHC class II is a monocyte and/or a B-cell.

In another aspect, the invention provides a method of producing a chimeric antigen receptor (CAR) T-cell. The method comprises contacting T-cells (e.g., isolated T-cells) with (i) a superantigen comprising Staphylococcal enterotoxin A or an immunologically reactive variant and/or fragment thereof, and/or (ii) a cell comprising a major histocompatibility complex (MHC) class II, wherein the T-cells have been modified to comprise an exogenous nucleotide sequence encoding a chimeric antigen receptor (CAR). In certain embodiments, the superantigen comprises the amino acid sequence of SEQ ID NO: 3, or an immunologically reactive variant and/or fragment thereof. In certain embodiments, the cell comprising an MHC class II is an antigen presenting cell (APC). In certain embodiments, the cell comprising an MHC class II is a monocyte and/or a B-cell.

In another aspect, the invention provides (i) a T-cell (e.g., an isolated T-cell), (ii) a CAR T-cell (e.g., an isolated CAR-T cell), (iii) a population of T-cells (e.g., a population of isolated T-cells), or (iv) a population of CAR T-cells (e.g., a population of isolated CAR T-cells) produced by any of the foregoing methods. In another aspect, the invention provides a method of treating cancer in a subject in need thereof. The method comprises administering to the subject an effective amount of the foregoing T-cell or CAR T-cell or population of T-cells or CAR T-cells. In certain embodiments, the method further comprises administering to the subject an effective amount of a superantigen conjugate comprising a superantigen covalently linked to a targeting moiety that binds a first cancer antigen expressed by cancerous cells within the subject. In certain embodiments, the method does not comprise administering to the subject an effective amount of a superantigen conjugate comprising a superantigen covalently linked to a targeting moiety that binds a first cancer antigen expressed by cancerous cells within the subject.

In another aspect, the invention provides a pharmaceutical composition comprising T-cells (e.g., isolated T-cells), wherein at least 10% of the T-cells comprise a T-cell receptor comprising TRBV7-9. In certain embodiments, at least 20%, 30%, or 40% of the T-cells comprise a T-cell receptor comprising TRBV7-9. In another aspect, the invention provides a method of treating cancer in a subject in need thereof. The method comprises administering to the subject an effective amount of the foregoing pharmaceutical composition.

In another aspect, the invention provides a T-cell (e.g., an isolated T-cell) modified to have increased expression of TRBV7-9 relative to a T-cell that has not been modified. In certain embodiments, the T-cell comprises an exogenous nucleotide sequence encoding TRBV7-9. In certain emobdiments, the T-cell further comprises an exogenous nucleotide sequence encoding a chimeric antigen receptor (CAR). In another aspect, the invention provides a method of treating cancer in a subject in need thereof. The method comprises administering to the subject: (i) an effective amount of a superantigen conjugate comprising a superantigen covalently linked to a targeting moiety that binds a first cancer antigen expressed by cancerous cells within the subject; and/or (ii) an effective amount of the foregoing T-cell.

In certain embodiments of any of the foregoing methods of treating cancer, the cancer is selected from a cancer expressing 5T4, mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD47, CD49f, CD56, CD74, CD123, CD133, CD138, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-a and β (FRa and (3), Ganglioside G2 (GD2), Ganglioside G3 (GD3), an Epidermal Growth Factor Receptor (EGFR), Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Ra2), K-light chain, kinase insert domain receptor (KDR), Lewis A (CA19.9), Lewis Y (LeY), LI cell adhesion molecule (LICAM), melanoma-associated antigen 1 (melanoma antigen family A1, MAGE-A1), Mucin 16 (MUC-16), Mucin 1 (MUC-1), KG2D ligands, cancer-testis antigen NY-ESO-1, tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-IH, HERK-V, IL-1 IRa, Latent Membrane Protein 1 (LMP1), Neural cell-adhesion molecule (N-CAM/CD56), programmed cell death receptor ligand 1 (PD-L1), B Cell Maturation Antigen (BCMA), and Trail Receptor (TRAIL R), or any combination thereof. In certain embodiments, the cancer is selected from a cancer expressing 5T4, EpCAM, HER2, EGFRViii, and IL13Rα2, for example, the cancer is a 5T4-expressing cancer.

In certain embodiments of any of the foregoing methods of treating cancer, the cancer comprises a solid tumor. In certain embodiments, the cancer is selected from breast cancer, bladder cancer, cervical cancer, colon cancer, colorectal cancer, endometrial cancer, gastric cancer, head and neck cancer, liver cancer, melanoma, mesothelioma, non-small cell lung cancer, ovarian cancer, pancreatic cancer, prostate cancer, renal cell cancer, and skin cancer.

These and other aspects and features of the invention are described in the following detailed description and claims.

### DESCRIPTION OF THE DRAWINGS

The invention can be more completely understood with reference to the following drawings.

FIG. 1 is a sequence alignment showing the homologous A-E regions in certain wild type and modified superantigens.

FIG. 2 is an amino acid sequence corresponding to an exemplary superantigen conjugate, naptumomab estafenatox/ANYARA®, which comprises two protein chains. The first protein chain comprises residues 1 to 458 of SEQ ID NO: 7 (see also, SEQ ID NO: 8), and includes a chimeric 5T4 Fab heavy chain, corresponding to residues 1 to 222 of SEQ ID NO: 7, and the SEA/E-120 superantigen, corresponding to residues 226 to 458 of SEQ ID NO: 7, covalently linked via a GGP tripeptide linker, corresponding to residues 223-225 of SEQ ID NO: 7. The second chain comprises residues 459 to 672 of SEQ ID NO: 7 (see also, SEQ ID NO: 9) and includes a chimeric 5T4 Fab light chain. The two protein chains are held together by non-covalent interactions between the Fab heavy and light chains.

FIG. 3 is a schematic depiction of an exemplary superantigen conjugate, naptumomab estafenatox/ANYARA®.

FIG. 4 is a bar chart illustrating the effect of CAR T cells in combination with the tumor-targeted superantigen naptumomab estafenatox (“NAP”) on the viability of the head and neck tumor cell line FaDu. Viability of FaDu cells was measured following a 4 hour co-culture with either Her2 CAR T cells (“CAR T”) or negative control CAR T cells (“T cells”) in the presence or absence of NAP (0.1 ng/ml). Viability was normalized to an untreated control (“no T cells”). Results are shown from left to right for: untreated control (“no T cells”); negative control CAR T cells (“T cells”) without NAP; negative control CAR T cells (“T cells”) with 0.1 ng/ml NAP; Her2 CAR T cells electroplated with 0.25 μg of CAR mRNA (“CAR T”) without NAP; and Her2 CAR T cells electroplated with 0.25 μg of CAR mRNA (“CAR T”) with 0.1 ng/ml NAP. Mean±SD; one-way ANOVA (*** p=0.0007 vs. control, **** p<0.0001 vs. all test groups, NS=not significant); #=CAR T cells grown in the presence of αCD3 and αCD28 antibodies; ^{&}=CART cells or T cells grown in the presence of NAP.

FIG. 5 illustrates the effect of different CAR T cell activation methods on CAR expression. Expression of a myc-tagged CAR in activated CAR T cells was analyzed by flow cytometry. The table shows mean fluorescence intensity (MFI), indicative of CAR expression, following the indicated activation method.

FIG. 6 illustrates the percentage of TRBV7-9-expressing CD8⁺ T cells grown under the indicated activation conditions. TRBV7-9 was stained with a multimer of NAP-PE and analyzed by flow cytometry.

FIG. 7 is a bar chart illustrating the effect of different CAR T cell activation methods on CAR T cell activity, as measured by the viability of the head and neck tumor cell line FaDu following CAR T cell treatment. The survival rates of FaDu cells were measured following 4 hour co-culture with Her2 CAR T cells that had been activated by the indicated method. Survival (viability) was normalized to an untreated control (“no CAR T cells”). Results are shown from left to right for: untreated control (“no CAR T cells”); CAR T cells grown in the presence of αCD3 and IL2; CAR T cells grown in the presence of αCD3, αCD28, and IL2; CAR T cells grown in the presence of NAP (1 μg/ml) and IL2; and CAR T cells grown in the presence of NAP (10 μg/ml) and IL2. n=4; mean±SD; one-way ANOVA (**** p<0.0001 vs CD3 or CD3/CD28).

FIG. 8 illustrates the effect of different CAR T cell activation methods on expression of INFγ and the degranulation marker CD107a. FaDu tumor cells were incubated with CD8⁺ CAR T cells activated by the indicated method for 4 hours. Control T cells were incubated alone without any target cells. Thereafter, CD8⁺ CAR T cells were stained and analyzed for INFγ and CD107a expression by flow cytometry (FIG. 8A). The percentage of CD8+ CAR T cells expressing IFNγ (FIG. 8B, left) and CD107a (FIG. 8B, right) is presented. Results are shown from left to right for: CAR T cells grown in the presence of aCD3 and IL2; CAR T cells grown in the presence of aCD3, aCD28, and IL2; CAR T cells grown in the presence of NAP (1 μg/ml) and IL2; and CAR T cells grown in the presence of NAP (10 μg/ml) and IL2.

FIG. 9 is a bar chart illustrating the effect of CAR T cells in combination with either NAP or unconjugated Staphylococcal enterotoxin superantigen (SEA) on the viability of the head and neck tumor cell line FaDu. The survival rates of FaDu cells were measured following 4 hour co-culture with Her2 CAR T cells that had been activated by the indicated method. Survival (viability) was normalized to an untreated control. Results are shown from left to right for: no T cell treatment (“control”); CAR T cells without NAP or SEA (“CAR T”); CAR T cells with 0.01 ng/ml NAP (“CAR T +NAP”); CAR T cells with 0.01 ng/ml SEA (“CAR T +SEA”). Mean±SD; one-way ANOVA (**** p<0.0001 vs. all test groups, NS=not significant); #=CAR T cells grown in the presence of αCD3 and αCD28 antibodies; ^{&}=CAR T cells grown in the presence of 10 μg/ml NAP; ^{λ}=CAR T cells grown in the presence of 10 ng/ml SEA.

### DETAILED DESCRIPTION

The invention is based, in part, upon the discovery that a targeted immune response against a cancer in a subject can be enhanced by combining a superantigen conjugate comprising a superantigen (e.g., engineered Staphylococcal enterotoxin superantigen SEA/E-120) covalently linked to a targeting moiety that binds a cancer antigen with an immune cell (e.g., a T-cell, e.g., a chimeric antigen receptor (CAR) T-cell). Furthermore, it has been discovered that an anti-cancer treatment using a superantigen conjugate and immune cell can be enhanced by using immune cells that express T-cell receptors that bind to the superantigen (e.g., T-cell receptors comprising T-cell receptor β variable 7-9).

Accordingly, in one aspect, the invention provides a method of treating cancer in a subject in need thereof. The method comprises administering to the subject: (i) an effective amount of a superantigen conjugate comprising a superantigen covalently linked to a targeting moiety that binds a first cancer antigen expressed by cancerous cells within the subject; and (ii) an effective amount of an immune cell (e.g., an isolated immune cell) comprising an exogenous nucleotide sequence encoding a chimeric antigen receptor (CAR) that binds a second cancer antigen expressed by cancerous cells within the subject.

In another aspect, the invention provides a pharmaceutical composition comprising: (i) a superantigen conjugate comprising a superantigen covalently linked to a targeting moiety that binds a first cancer antigen expressed by cancerous cells within the subject; (ii) an immune cell (e.g., an isolated immune cell) comprising an exogenous nucleotide sequence encoding a chimeric antigen receptor (CAR) that binds a second cancer antigen expressed by cancerous cells within the subject; and (iii) a pharmaceutically acceptable carrier or diluent. In another aspect, the invention provides a method of treating cancer in a subject in need thereof. The method comprises administering to the subject an effective amount of the foregoing pharmaceutical composition.

In another aspect, the invention provides a method of expanding T-cells (e.g., isolated T-cells) comprising a T-cell receptor comprising TRBV7-9. The method comprises contacting the T-cells with (i) a superantigen comprising Staphylococcal enterotoxin A or an immunologically reactive variant and/or fragment thereof, and/or (ii) a cell comprising a major histocompatibility complex (MHC) class II.

In another aspect, the invention provides a method of producing a T-cell (e.g., an isolated T-cell) for use in the treatment of a subject. The method comprises contacting T-cells (e.g., T-cells isolated from the subject) with (i) a superantigen comprising Staphylococcal enterotoxin A or an immunologically reactive variant and/or fragment thereof, and/or (ii) a cell comprising a major histocompatibility complex (MHC) class II.

In another aspect, the invention provides a method of producing a chimeric antigen receptor (CAR) T-cell. The method comprises: (a) contacting T-cells (e.g., T-cells isolated from a subject) with (i) a superantigen comprising Staphylococcal enterotoxin A or an immunologically reactive variant and/or fragment thereof, and/or (ii) a cell comprising a major histocompatibility complex (MHC) class II; and (b) modifying the T-cells to comprise an exogenous nucleotide sequence encoding a chimeric antigen receptor (CAR).

In another aspect, the invention provides a method of producing a chimeric antigen receptor (CAR) T-cell. The method comprises: (a) modifying T-cells (e.g., T-cells isolated from a subject) to comprise an exogenous nucleotide sequence encoding a chimeric antigen receptor (CAR); and (b) contacting the T-cells with (i) a superantigen comprising Staphylococcal enterotoxin A or an immunologically reactive variant and/or fragment thereof, and/or (ii) a cell comprising a major histocompatibility complex (MHC) class II.

In another aspect, the invention provides a method of producing a chimeric antigen receptor (CAR) T-cell. The method comprises modifying T-cells (e.g., isolated T-cells) to comprise an exogenous nucleotide sequence encoding a chimeric antigen receptor (CAR), wherein the T-cells have been contacted with (i) a superantigen comprising Staphylococcal enterotoxin A or an immunologically reactive variant and/or fragment thereof, and/or (ii) a cell comprising a major histocompatibility complex (MHC) class II.

In another aspect, the invention provides a method of producing a chimeric antigen receptor (CAR) T-cell. The method comprises contacting T-cells (e.g., isolated T-cells) with (i) a superantigen comprising Staphylococcal enterotoxin A or an immunologically reactive variant and/or fragment thereof, and/or (ii) a cell comprising a major histocompatibility complex (MHC) class II, wherein the T-cells have been modified to comprise an exogenous nucleotide sequence encoding a chimeric antigen receptor (CAR).

In another aspect, the invention provides a T-cell (e.g., an isolated T-cell) or CAR T-cell (e.g., an isolated CAR T-cell) produced by any of the foregoing methods. In another aspect, the invention provides a population of T-cells (e.g., a population of isolated T-cells) or a population of CAR T-cells (e.g., a population of isolated CAR T-cells) produced by any of the foregoing methods. In another aspect, the invention provides a method of treating cancer in a subject in need thereof. The method comprises administering to the subject an effective amount of the foregoing T-cell or CAR T-cell or population of T-cells or CAR T-cells. In certain embodiments, the method further comprises administering to the subject an effective amount of a superantigen conjugate comprising a superantigen covalently linked to a targeting moiety that binds a first cancer antigen expressed by cancerous cells within the subject. In certain embodiments, the method does not comprise administering to the subject an effective amount of a superantigen conjugate comprising a superantigen covalently linked to a targeting moiety that binds a first cancer antigen expressed by cancerous cells within the subject.

In another aspect, the invention provides a pharmaceutical composition comprising T-cells (e.g., isolated T-cells), wherein at least 10% of the T-cells comprise a T-cell receptor comprising TRBV7-9. In another aspect, the invention provides a method of treating cancer in a subject in need thereof. The method comprises administering to the subject an effective amount of the foregoing pharmaceutical composition.

In another aspect, the invention provides a T-cell (e.g., an isolated T-cell) modified to have increased expression of TRBV7-9 relative to a T-cell that has not been modified. In certain embodiments, the T-cell comprises an exogenous nucleotide sequence encoding TRBV7-9. In another aspect, the invention provides a method of treating cancer in a subject in need thereof. The method comprises administering to the subject: (i) an effective amount of a superantigen conjugate comprising a superantigen covalently linked to a targeting moiety that binds a first cancer antigen expressed by cancerous cells within the subject; and/or (ii) an effective amount of the foregoing T-cell.

Various features and aspects of the invention are discussed in more detail below.

### I. Definitions

Unless defined otherwise, technical and scientific terms used herein have the same meaning as commonly understood by one of ordinary skill in the art to which this invention belongs. For purposes of the present invention, the following terms are defined below.

As used herein, the terms “a” or “an” may mean one or more. For example, a statement such as “treatment with a superantigen and an immune cell,” can mean treatment: with one superantigen and immune cell; with more than one superantigen and one immune cell; with one superantigen and more than one immune cell; or with more than one superantigen and more than one immune cell.

As used herein, unless otherwise indicated, the term “antibody” is understood to mean an intact antibody (e.g., an intact monoclonal antibody) or antigen-binding fragment of an antibody, including an intact antibody or antigen-binding fragment of an antibody (e.g., a phage display antibody including a fully human antibody, a semisynthetic antibody or a fully synthetic antibody) that has been optimized, engineered or chemically conjugated. Examples of antibodies that have been optimized are affinity-matured antibodies. Examples of antibodies that have been engineered are Fc optimized antibodies, antibodies engineered to reduce immunogenicity, and multi-specific antibodies (e.g., bispecific antibodies). Examples of antigen-binding fragments include Fab, Fab′, F(ab′)₂, Fv, single chain antibodies (e.g., scFv), minibodies and diabodies. An antibody conjugated to a toxin moiety is an example of a chemically conjugated antibody.

As used herein, the terms “cancer” and “cancerous” are understood to mean the physiological condition in mammals that is typically characterized by unregulated cell growth. Examples of cancer include, but are not limited to, melanoma, carcinoma, lymphoma, blastoma, sarcoma, and leukemia or lymphoid malignancies. More particular examples of cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, bone cancer, brain cancer, retinoblastoma, endometrial cancer or uterine carcinoma, salivary gland carcinoma, kidney or renal cancer, prostate cancer, vulval cancer, thyroid cancer, hepatic carcinoma, anal carcinoma, penile carcinoma, testicular cancer, as well as head and neck cancer, gum or tongue cancer. The cancer comprises cancer or cancerous cells, for example, the cancer may comprise a plurality of individual cancer or cancerous cells, for example, a leukemia, or a tumor comprising a plurality of associated cancer or cancerous cells.

As used herein, the term “refractory” refers to a cancer that does not respond or no longer responds to a treatment. In certain embodiments, a refractory cancer can be resistant to a treatment before or at the beginning of the treatment. In other embodiments, the refractory cancer can become resistant during or after a treatment. A refractory cancer is also called a resistant cancer. As used herein, the term “recurrence” or “relapse” refers to the return of a refractory cancer or the signs and symptoms of a refractory cancer after a positive response a prior treatment (e.g., a reduction in tumor burden, a reduction in tumor volume, a reduction in tumor metastasis, or a modulation of a biomarker indicative of a positive response to a treatment).

As used herein, the term “immunogen” is a molecule that provokes (evokes, induces, or causes) an immune response. This immune response may involve antibody production, the activation of certain cells, such as, for example, specific immunologically-competent cells, or both. An immunogen may be derived from many types of substances, such as, but not limited to, molecules from organisms, such as, for example, proteins, subunits of proteins, killed or inactivated whole cells or lysates, synthetic molecules, and a wide variety of other agents both biological and nonbiological. It is understood that essentially any macromolecule (including naturally occurring macromolecules or macromolecules produced via recombinant DNA approaches), including virtually all proteins, can serve as immunogens.

As used herein, the term “immunogenicity” relates to the ability of an immunogen to provoke (evoke, induce, or cause) an immune response. Different molecules may have differing degrees of immunogenicity, and a molecule having an immunogenicity that is greater compared to another molecule is known, for example, to be capable of provoking (evoking, inducing, or causing) a greater immune response than would an agent having a lower immunogenicity.

As used herein, the term “antigen” as used herein refers to a molecule that is recognized by antibodies, specific immunologically-competent cells, or both. An antigen may be derived from many types of substances, such as, but not limited to, molecules from organisms, such as, for example, proteins, subunits of proteins, nucleic acids, lipids, killed or inactivated whole cells or lysates, synthetic molecules, and a wide variety of other agents both biological and non-biological.

As used herein, the term “antigenicity” relates to the ability of an antigen to be recognized by antibodies, specific immunologically-competent cells, or both.

As used herein, the term “epitope spreading” refers to the diversification of the epitope specificity of an immune response from an initial epitope-specific immune response directed against an antigen to other epitopes on that antigen (intramolecular spreading) or other antigens (intermolecular spreading). Epitope spreading allows a subject's immune system to determine additional target epitopes not initially recognized by the immune system in response to the original therapeutic protocol while reducing the possibility of escape variants in a tumor population and thus affect progression of disease.

As used herein, the term “immune response” refers to a response by a cell of the immune system, such as a B cell, T cell (CD4+ or CD8+), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), and refers to a response by a CD4+ T cell, CD8+ T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response.

As used herein, the term “major histocompatibility complex,” or “MHC,” refers to a specific cluster of genes, many of which encode evolutionarily related cell surface proteins involved in antigen presentation, that are important determinants of histocompatibility. Class I MEW, or MHC-I, function mainly in antigen presentation to CD8⁺ T lymphocytes (CD8⁺ T-Cells). Class II MEW, or MHC-II, function mainly in antigen presentation to CD4⁺ T lymphocytes (CD4⁺ T-Cells).

As used herein, the term “derived,” for example “derived from,” includes, but is not limited to, for example, wild-type molecules derived from biological hosts such as bacteria, viruses and eukaryotic cells and organisms, and modified molecules, for example, modified by chemical means or produced in recombinant expression systems.

As used herein, the terms “seroreactive,” “seroreaction” or “seroreactivity” are understood to mean the ability of an agent, such as a molecule, to react with antibodies in the serum of a mammal, such as, but not limited to, a human. This includes reactions with all types of antibodies, including, for example, antibodies specific for the molecule and nonspecific antibodies that bind to the molecule, regardless of whether the antibodies inactivate or neutralize the agent. As is known in the art, different agents may have different seroreactivity relative to one another, wherein an agent having a seroreactivity lower than another would, for example, react with fewer antibodies and/or have a lower affinity and/or avidity to antibodies than would an agent having a higher seroreactivity. This may also include the ability of the agent to elicit an antibody immune response in an animal, such as a mammal, such as a human.

As used herein, the terms “soluble T-cell receptor,” or “soluble TCR,” are understood to mean a “soluble” T-cell receptor comprising the chains of a full-length (e.g., membrane bound) receptor, except that the transmembrane region of the receptor chains are deleted or mutated so that the receptor, when expressed by a cell, will not insert into, traverse or otherwise associate with the membrane. A soluble T-cell receptor may comprise only the extracellular domains or extracellular fragments of the domains of the wild-type receptor (e.g., lacks the transmembrane and cytoplasmic domains).

As used herein, the term “superantigen” is understood to mean a class of molecules that stimulate a subset of T-cells by binding to MHC class II molecules and VP domains of T-cell receptors, thereby activating T-cells expressing particular VP gene segments. The term includes wild-type, naturally occurring superantigens, for example, those isolated from certain bacteria or expressed from unmodified genes from same, as well as modified superantigens, wherein, for example, the DNA sequence encoding a superantigen has been modified, for example, by genetic engineering, to, for example, produce a fusion protein with a targeting moiety, and/or alter certain properties of the superantigen, such as, but not limited to, its MHC class II binding (for example, to reduce affinity) and/or its seroreactivity, and/or its immunogenicity, and/or antigenicity (for example, to reduce its seroreactivity). The definition includes wild-type and modified superantigens and any immunologically reactive variants and/or fragments thereof described herein or in the following U.S. patents and patent applications: U.S. Pat. Nos. 5,858,363, 6,197,299, 6,514,498, 6,713,284, 6,692,746, 6,632,640, 6,632,441, 6,447,777, 6,399,332, 6,340,461, 6,338,845, 6,251,385, 6,221,351, 6,180,097, 6,126,945, 6,042,837, 6,713,284, 6,632,640, 6,632,441, 5,859,207, 5,728,388, 5,545,716, 5,519,114, 6,926,694, 7,125,554, 7,226,595, 7,226,601, 7,094,603, 7,087,235, 6,835,818, 7,198,398, 6,774,218, 6,913,755, 6,969,616, and 6,713,284, U.S. Patent Application Nos. 2003/0157113, 2003/0124142, 2002/0177551, 2002/0141981, 2002/0115190, 2002/0051765, and 2001/0046501, and PCT International Publication Number WO/03/094846.

As used herein, the term “targeting moiety” refers to any structure, molecule or moiety that is able to bind to a cellular molecule, for example, a cell surface molecule, preferably a disease specific molecule such as an antigen expressed preferentially on a cancer (or cancerous) cell. Exemplary targeting moieties include, but are not limited to, antibodies (including antigen binding fragments thereof) and the like, soluble T-cell receptors, interleukins, hormones, and growth factors.

As used herein, the terms “tumor-targeted superantigen” or “TTS” or “cancer-targeted superantigen” are understood to mean a molecule comprising one or more superantigens covalently linked (either directly or indirectly) with one or more targeting moieties.

As used herein, the term “T-cell receptor” is understood to mean a receptor that is specific to T-cells, and includes the understanding of the term as known in the art. The term also includes, for example, a receptor that comprises a disulfide-linked heterodimer of the highly variable α or β chains expressed at the cell membrane as a complex with the invariant CD3 chains, and a receptor made up of variable γ and δ chains expressed at the cell membrane as a complex with CD3 on a subset of T-cells.

As used herein, the terms “therapeutically effective amount” and “effective amount,” are understood to mean an amount of an active agent, for example, a pharmaceutically active agent or a pharmaceutical composition that produces at least some effect in treating a disease or a condition. The effective amount of pharmaceutically active agent(s) used to practice the present invention for a therapeutic treatment varies depending upon the manner of administration, the age, body weight, and general health of the subject. An effective amount can be administered in one or more administrations, applications or dosages and is not intended to be limited to a particular formulation or administration route.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

As used herein, the terms “treat,” “treating” and “treatment” are understood to mean the treatment of a disease in a mammal, e.g., in a human. This includes: (a) inhibiting the disease, i.e., arresting its development; and (b) relieving the disease, i.e., causing regression of the disease state; and (c) curing the disease. As used in the context of a therapeutic treatment, the terms “prevent” or “block” are understood to completely prevent or block, or not completely prevent or block (e.g., partially prevent or block) a given act, action, activity, or event.

As used herein, the term “inhibits the growth of a cancer” is understood to mean a measurably slowing, stopping, or reversing the growth rate of the cancer or cancerous cells in vitro or in vivo. Desirably, the growth rate is slowed by 20%, 30%, 50%, or 70% or more, as determined using a suitable assay for determination of cell growth rates. Typically, a reversal of growth rate is accomplished by initiating or accelerating necrotic or apoptotic mechanisms of cell death in neoplastic cells, resulting in a shrinkage of a neoplasm.

As used herein, the terms “variant,” “variants,” “modified,” “altered,” “mutated,” and the like, are understood to mean proteins or peptides and/or other agents and/or compounds that differ from a reference protein, peptide or other compound. Variants in this sense are described below and elsewhere in greater detail. For example, changes in a nucleic acid sequence of the variant may be silent, e.g., they may not alter the amino acids encoded by the nucleic acid sequence. Where alterations are limited to silent changes of this type a variant will encode a peptide with the same amino acid sequence as the reference peptide. Changes in the nucleic acid sequence of the variant may alter the amino acid sequence of a peptide encoded by the reference nucleic acid sequence. Such nucleic acid changes may result in amino acid substitutions, additions, deletions, fusions and/or truncations in the protein or peptide encoded by the reference sequence, as discussed below. Generally, differences in amino acid sequences are limited so that the sequences of the reference and the variant are similar overall and, in many regions, identical. A variant and reference protein or peptide may differ in amino acid sequence by one or more substitutions, additions, deletions, fusions and/or truncations, which may be present in any combination. A variant may also be a fragment of a protein or peptide of the invention that differs from a reference protein or peptide sequence by being shorter than the reference sequence, such as by a terminal or internal deletion. Another variant of a protein or peptide of the invention also includes a protein or peptide which retains essentially the same function or activity as the reference protein or peptide. A variant may also be: (i) one in which one or more of the amino acid residues are substituted with a conserved or non-conserved amino acid residue and such substituted amino acid residue may or may not be one encoded by the genetic code, or (ii) one in which one or more of the amino acid residues includes a substituent group, or (iii) one in which the mature protein or peptide is fused with another compound, such as a compound to increase the half-life of the protein or peptide (for example, polyethylene glycol), or (iv) one in which the additional amino acids are fused to the mature protein or peptide, such as a leader or secretory sequence or a sequence which is employed for purification of the mature protein or peptide. Variants may be made by mutagenesis techniques, and/or altering mechanisms such as chemical alterations, fusions, adjuncts and the like, including those applied to nucleic acids, amino acids, cells or organisms, and/or may be made by recombinant means.

As used herein, the term “sequential dosage” and related terminology refers to the administration of at least one agent (e.g., a superantigen conjugate), with at least one additional agent (e.g., an immune cell), and includes staggered doses of these agents (i.e., time-staggered) and variations in dosage amounts. This includes one agent being administered before, overlapping with (partially or totally), or after administration of another agent. In addition, the term “sequential dosage” and related terminology also includes the administration of at least one superantigen, one immune cell and more or more optional additional compounds such as, for example, a corticosteroid, an immune modulator, and another agent designed to reduce potential immunoreactivity to the superantigen conjugate administered to the subject.

As used herein, the terms “systemic” and “systemically” in the context of administration are understood to mean administration of an agent such that the agent is exposed to at least one system associated with the whole body, such as but not limited to the circulatory system, immune system, and lymphatic system, rather than only to a localized part of the body, such as but not limited to within a tumor. Thus, for example, a systemic therapy or an agent administered systematically is a therapy or an agent in which at least one system associated with the entire body is exposed to the therapy or agent, as opposed to, rather than just a target tissue.

As used herein, the term “parenteral administration” includes any form of administration in which the compound is absorbed into the subject without involving absorption via the intestines. Exemplary parenteral administrations that are used in the present invention include, but are not limited to intramuscular, intravenous, intraperitoneal, or intraarticular administration.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

At various places in the present specification, values are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### II. Immune Cells

Among other things, the invention provides (i) methods and compositions comprising an immune cell useful in the treatment of cancer, where the immune cell can be used as is or in combination with a superantigen conjugate, and (ii) methods of making an immune cell useful in the treatment of cancer.

Immune cells include, e.g., lymphocytes, such as B-cells and T-cells, natural killer cells (NK-cells), natural killer T-cells (NKT-cells), myeloid cells, such as monocytes, macrophages, eosinophils, mast cells, basophils, and granulocytes.

In certain embodiments, the immune cell is a T-cell, which can be, for example, a cultured T-cell, e.g., a primary T-cell, or a T-cell from a cultured T-cell line, e.g., Jurkat, SupTi, etc., or a T-cell obtained from a mammal, for example, from a subject to be treated. If obtained from a mammal, the T-cell can be obtained from numerous sources, including but not limited to blood, bone marrow, lymph node, the thymus, or other tissues or fluids. T-cells can also be enriched or purified. The T-cell can be any type of T-cell and can be of any developmental stage, including but not limited to, CD4+/CD8+ double positive T-cells, CD4+ helper T-cells, e.g., Th1 and Th2 cells, CD4+ T-cells, CD8+ T-cells (e.g., cytotoxic T-cells), tumor infiltrating lymphocytes (TILs), memory T-cells (e.g., central memory T-cells and effector memory T-cells), naive T-cells, and the like. The cells (e.g., the T-cells) can include autologous cells derived from a subject to be treated, or alternatively allogenic cells derived from a donor.

In certain embodiments, the T-cell binds an antigen, e.g., a cancer antigen, through a T-cell receptor. The T-cell receptor may be an endogenous or a recombinant T-cell receptor. T-cell receptors comprise two chains referred to as the α- and β-chains, that combine on the surface of a T-cell to form a heterodimeric receptor that can recognize MHC-restricted antigens. Each of α-and β-chain comprises two regions, a constant region and a variable region. Each variable region of the α- and β-chains defines three loops, referred to as complementary determining regions (CDRs) known as CDR₁, CDR₂, and CDR₃ that confer the T-cell receptor with antigen binding activity and binding specificity.

In certain embodiments, the immune cell comprises a T-cell receptor comprising T-cell receptor β variable 7-9 (TRBV7-9). An exemplary amino acid sequence of TRBV7-9 is depicted in SEQ ID NO: 11, and an exemplary nucleotide sequence encoding TRBV7-9 is depicted in SEQ ID NO: 12. The term TRBV7-9 includes variants having one or more amino acid substitutions, deletions, or insertions relative to wild-type TRBV7-9 sequence, and/or fusion proteins or conjugates including TRBV7-9. As used herein, the term “functional fragment” of TRBV7-9 refers to a fragment of full-length TRBV7-9 that retains, for example, at least 10%, at least 20%, at least 30%, at least 40%, at least 50%, at least 60%, at least 70%, at least 80%, at least 90%, or 100% of the SEA/E-120 binding activity of the corresponding full-length, naturally occurring TRBV7-9.

It is contemplated that, in a pharmaceutical composition comprising immune cells, e.g., T-cells, comprising a T-cell receptor comprising T-cell receptor β variable 7-9 (TRBV7-9), at least about 2%, at least about 5%, at least about 10%, at least about 20%, at least about 30%, at least about 40%, at least about 50%, at least about 60%, at least about 70%, at least about 80%, at least about 90%, or about 100% of the cells may comprise a T-cell receptor comprising TRBV7-9. For example, in certain embodiments, from about 2% to about 100%, from about 5% to about 100%, from about 10% to about 100%, from about 20% to about 100%, from about 30% to about 100%, from about 40% to about 100%, from about 60% to about 100%, from about 80% to about 100%, from about 2% to about 80%, from about 5% to about 80%, from about 10% to about 80%, from about 20% to about 80%, from about 30% to about 80%, from about 40% to about 80%, from about 60% to about 80%, from about 2% to about 60%, from about 5% to about 60%, from about 10% to about 60%, from about 20% to about 60%, from about 30% to about 60%, from about 40% to about 60%, from about 2% to about 40%, from about 5% to about 40%, from about 10% to about 40%, from about 20% to about 40%, from about 30% to about 40%, from about 2% to about 30%, from about 5% to about 30%, from about 10% to about 30%, from about 20% to about 30%, from about 2% to about 20%, from about 5% to about 20%, from about 10% to about 20%, from about 2% to about 10%, from about 5% to about 10%, or from about 2% to about 5% of the cells comprise a T-cell receptor comprising TRBV7-9.

In certain embodiments, the immune cell, e.g., T-cell or NKT-cell, binds to an antigen, e.g., a cancer antigen, through a chimeric antigen receptor (CAR), i.e., the T-cell or NKT-cell comprises an exogenous nucleotide sequence encoding a CAR. As used herein, the terms “chimeric antigen receptor,” or “CAR,” refer to any artificial receptor including an antigen-specific binding moiety and one or more signaling chains derived from an immune receptor. CARs can comprise a single chain fragment variable (scFv) of an antibody specific for an antigen coupled via hinge and transmembrane regions to cytoplasmic domains of T-cell signaling molecules (e.g. a T-cell costimulatory domain (e.g., from CD28, CD137, OX40, ICOS, or CD27) in tandem with a T-cell triggering domain (e.g. from CD3δ)) and/or to cytoplasmic domains of NK-cell signaling molecules (e.g. DNAX-activation protein 12 (DAP12)). A T-cell expressing a chimeric antigen receptor is referred to as a CAR T-cell, an NK-cell expressing a chimeric antigen receptor is referred to as a CAR NK-cell, and an NKT-cell expressing a chimeric antigen receptor is referred to as a CAR NKT-cell.

Exemplary CAR T-cells include CD19 targeted CTL019 cells (Novartis; see, Grupp et al. (2015) BLOOD 126:4983), JCAR014 (Juno Therapeutics), JCAR015/19-28z cells (Juno Therapeutics; see, Park et al. (2015) J. CLIN. ONCOL. 33(15S):7010), JCAR017 cells (Juno Therapeutics), KTE-C19 cells (Kite Pharma; see, Locke et al. (2015) BLOOD 126:3991), and UCART19 cells (Cellectis; see, Gouble et al. (2014) BLOOD 124:4689). Additional exemplary CD19 targeted CARs or CD19 targeted CAR T-cells are described in U.S. Pat. Nos. 7,446,179, 8,399,645, U.S. Patent Publication Nos. US20130071414, US20140370045, US20140271635, US20170166623, US20150283178, and US20170107286, International (PCT) Publication Nos. WO2009091826, WO2012079000, WO2014153270, WO2014184143, WO2015095895, WO2016210293, WO2016139487, and WO2016100232, and Makita et al. (2017) CANCER SCIENCE 108(6):1109-1118, Brentjens et al. (2011) BLOOD 118(18):4817, Davila et al. (2014) SCI. TRANSL. MED. 6(224):224, Lee et al. (2015) LANCET 385(9967):517, Brentjens et al. (2013) SCI. TRANSL. MED. 5(177):177, Grupp et al. (2013) N. ENGL. J. MED. 368(16):1509, Porter et al. (2011) N. ENGL. J. MED. 365(8):725, Kochenderfer et al. (2013) BLOOD, and Kalos et al. (2011) SCI. TRANSL. MED. 3(95):95. Exemplary mesothelin targeted CAR T-cells are described in International (PCT) Publication Nos. WO2013142034, WO2015188141, and WO2017040945. Additional exemplary CARs or CAR T-cells are described in U.S. Pat. Nos. 5,712,149, 5,906,936, 5,843,728, 6,083,751, 6,319,494, 7,446,190, 7,741,965, 8,399,645, 8,906,682, 9,181,527, 9,272,002, and 9,266,960, U.S. Patent Publication Nos. US20160362472, US20160200824, and US20160311917 and International (PCT) Publication No. WO2015120180. Engineered immune cells containing a T-cell receptor knockout and a chimeric antigen receptor that binds CD123 are described in International (PCT) Publication No. WO2016120220.

CAR T-cells may be generated using methods known in the art. T-cells can be obtained from a number of sources, including peripheral blood mononuclear cells, bone marrow, lymph node tissue, cord blood, thymus tissue, tissue from a site of infection, ascites, pleural effusion, spleen tissue, tumors, and I-cell lines. For example, T-cells can be obtained from a unit of blood collected from a subject using any number of techniques known to the skilled artisan, such as Ficoll™ separation. In certain embodiments, cells from the circulating blood of an individual are obtained by apheresis. The apheresis product typically contains lymphocytes, including T-cells, monocytes, granulocytes, B cells, other nucleated white blood cells, red blood cells, and platelets. Cells collected by apheresis may be washed to remove the plasma fraction and to place the cells in an appropriate buffer or media for subsequent processing steps. For example, the cells may be washed with phosphate buffered saline (PBS). After washing, the cells may be resuspended in a variety of biocompatible buffers, such as, for example, Ca2+-free or Mg2+-free PBS, PlasmaLyte A, or other saline and/or buffer solutions. T-cells may also be isolated from peripheral blood lymphocytes by lysing red blood cells and depleting monocytes, for example, by centrifugation through a PERCOLL™ gradient or by counterflow centrifugal elutriation. A specific subpopulation of T-cells, such as CD3+, CD28+, CD4+, CD8+, CD45RA+, and CD45RO+ T-cells, can be further isolated by positive or negative selection techniques. For example, in one embodiment, T-cells are isolated by incubation with anti-CD³/ₐnti-CD28-conjugated beads, such as DYNABEADS® M-450 CD3/CD28 (Thermo Fisher Scientific), for a time period sufficient for positive selection of the desired T-cells.

17-cells may be engineered to express CARS by methods known in the art. Generally, a polynucleotide vector is constructed that encodes the CAR and the vector is transfected or transduced into a population of T-cells. For example, a nucleotide sequence encoding a CAR can be delivered into cells using a retroviral or lentiviral vector. An exemplary retroviral vector includes, but is not limited to, the vector backbone pMSGV1-CD8-28BBZ, which is derived from pMSGV (murine stem cell virus-based splice-gag vector). For other exemplary lentiviral vectors see, for example, Dull et at., (1998) J. Virol 72:8463-8471, and U.S. Pat. Nos. 5,994,136, 6,682,907, 7,629,153, 8,329,462, 8,748,169, 9,101,584. Retroviral transduction may be performed using known techniques, such as that of Johnson e (Blood 1 14, 535-546 (2009)). The surface expression of a CAR on transduced T-cells may be determined, for example, by flow cytometry. A nucleotide sequence encoding a CAR can also be delivered into cells using in vitro transcribed mRNA.

T-cells and/or T-cells engineered to express CARs can be activated and expanded generally using methods as described, for example, in U.S. Pat. Nos, 6,352,694; 6,534,055; 6,905,680; 6,692,964; ,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. Generally, T-cells are expanded by contact with an agent that stimulates a CD3/TCR complex associated signal and a ligand that stimulates a co-stimulatory molecule on the surface of the T-cells. For example, T-cell populations may be stimulated by contact with an anti-CD3 antibody, anti-CD28 antibody, an anti-CD2 antibody, or a protein kinase C activator (e.g., bryostatin) and/or a calcium ionophore.

Further methods for manufacturing CAR T-cells are described, for example, in Levine et al. (2016) MOL. THER. METHODS CLIN. DEV. 4:92-101.

In certain embodiments, a CAR binds a cancer antigen selected from 5T4, mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD47, CD49f, CD56, CD74, CD123, CD133, CD138, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-a and β(FRa and β), Ganglioside G2 (GD2), Ganglioside G3 (GD3), an Epidermal Growth Factor Receptor (EGFR), Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Ra2), K-light chain, kinase insert domain receptor (KDR), Lewis A (CA19.9), Lewis Y (LeY), LI cell adhesion molecule (LICAM), melanoma-associated antigen 1 (melanoma antigen family A1, MAGE-A1), Mucin 16 (MUC-16), Mucin 1 (MUC-1), KG2D ligands, cancer-testis antigen NY-ESO-1, tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-IH, HERK-V, IL-1 IRa, Latent Membrane Protein 1 (LMP1), Neural cell-adhesion molecule (N-CAM/CD56), programmed cell death receptor ligand 1 (PD-L1), B Cell Maturation Antigen (BCMA), and Trail Receptor (TRAIL R).

### III. Superantigen Conjugate

### A. Superantigens

Superantigens are bacterial proteins, viral proteins, and human-engineered proteins, capable of activating T lymphocytes, for example, at picomolar concentrations. Superantigens can also activate large subsets of T lymphocytes (T-cells). Superantigens can bind to the major histocompatibility complex I (MHCI) without being processed and, in particular, can bind to conserved regions outside the antigen-binding groove on MEW class II molecules (e.g. on monocytes), avoiding most of the polymorphism in the conventional peptide-binding site. Superantigens can also bind to the Vβ chain of the T-cell receptor (TCR) rather than binding to the hypervariable loops of the T-cell receptor. Examples of bacterial superantigens include, but are not limited to, Staphylococcal enterotoxin (SE), Streptococcus pyogenes exotoxin (SPE), Staphylococcus aureus toxic shock-syndrome toxin (TSST-1), Streptococcal mitogenic exotoxin (SME), Streptococcal superantigen (SSA), Staphylococcal enterotoxin A (SEA), Staphylococcal enterotoxin A (SEB), and Staphylococcal enterotoxin E (SEE).

The polynucleotide sequences encoding many superantigens have been isolated and cloned and superantigens expressed from these or modified (reengineered) polynucleotide sequences have been used in anti-cancer therapy (see, naptumomab estafenatox/ANYARA®, discussed below). Superantigens expressed by these polynucleotide sequences may be wild-type superantigens, modified superantigens, or wild-type or modified superantigens conjugated or fused with targeting moieties. The superantigens may be administered to a mammal, such as a human, directly, for example by injection, or may be delivered, for example, by exposure of blood of a patient to the superantigen outside the body, or, for example, via placing a gene encoding a superantigen inside a mammal to be treated (e.g., via known gene therapy methods and vectors such as, for example, via cells containing, and capable of expressing, the gene) and expressing the gene within the mammal.

Examples of superantigens and their administration to mammals are described in the following U.S. patents and patent applications: U.S. Pat. Nos. 5,858,363, 6,197,299, 6,514,498, 6,713,284, 6,692,746, 6,632,640, 6,632,441, 6,447,777, 6,399,332, 6,340,461, 6,338,845, 6,251,385, 6,221,351, 6,180,097, 6,126,945, 6,042,837, 6,713,284, 6,632,640, 6,632,441, 5,859,207, 5,728,388, 5,545,716, 5,519,114, 6,926,694, 7,125,554, 7,226,595, 7,226,601, 7,094,603, 7,087,235, 6,835,818, 7,198,398, 6,774,218, 6,913,755, 6,969,616, and 6,713,284, U.S. Patent Application Nos. 2003/0157113, 2003/0124142, 2002/0177551, 2002/0141981, 2002/0115190, and 2002/0051765, and PCT International Publication Number WO/03/094846.

### B. Modified Superantigens

Within the scope of this invention, superantigens may be engineered in a variety of ways, including modifications that retain or enhance the ability of a superantigen to stimulate T lymphocytes, and may, for example, alter other aspects of the superantigen, such as, for example, its seroreactivity or immunogenicity. Modified superantigens include synthetic molecules that have superantigen activity (i.e., the ability to activate subsets of T lymphocytes).

It is contemplated that various changes may be made to the polynucleotide sequences encoding a superantigen without appreciable loss of its biological utility or activity, namely the induction of the T-cell response to result in cytotoxicity of the tumor cells. Furthermore, the affinity of the superantigen for the MEW class II molecule can be decreased with minimal effects on the cytotoxicity of the superantigen. This, for example, can help to reduce toxicity that may otherwise occur if a superantigen retains its wild-type ability to bind MEW class II antigens (as in such a case, class II expressing cells, such as immune system cells, could also be affected by the response to the superantigen).

Techniques for modifying superantigens (e.g., polynucleotides and polypeptides), including for making synthetic superantigens, are well known in the art and include, for example PCR mutagenesis, alanine scanning mutagenesis, and site-specific mutagenesis (see, U.S. Pat. Nos. 5,220,007; 5,284,760; 5,354,670; 5,366,878; 5,389,514; 5,635,377; and 5,789,166).

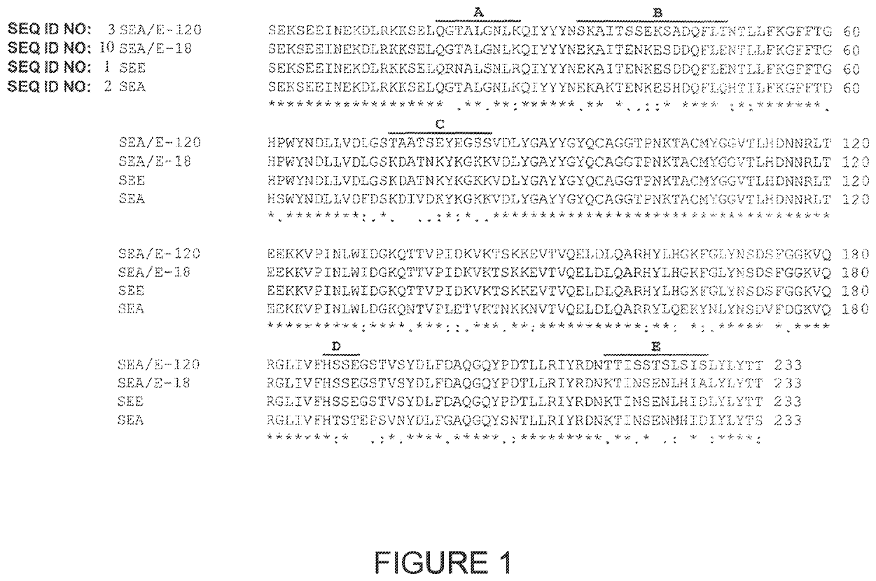

In some embodiments, a superantigen may be modified such that its seroreactivity is reduced compared to a reference wild-type superantigen, but its ability to activate T-cells is retained or enhanced relative to wild-type. One technique for making such modified superantigens includes substituting certain amino acids in certain regions from one superantigen to another. This is possible because many superantigens, including but not limited to, SEA, SEE, and SED, share sequence homology in certain areas that have been linked to certain functions (Marrack and Kappler (1990) SCIENCE 248(4959): 1066; see also FIG. 1, which shows region of homology between different wild type and engineered superantigens). For example, in certain embodiments of the present invention, a superantigen that has a desired T-cell activation-inducing response, but a non-desired high seroreactivity, is modified such that the resulting superantigen retains its T-cell activation ability but has reduced seroreactivity.

It is known and understood by those of skill in the art that the sera of humans normally contain various titers of antibodies against superantigens. For the staphylococcal superantigens, for instance, the relative titers are TSST-1>SEB>SEC-1>SE3>SEC2>SEA>SED>SEE. As a result, the seroreactivity of, for example, SEE (Staphylococcal enterotoxin E) is lower than that of, for example, SEA (Staphylococcal enterotoxin A). Based on this data, one skilled in the art may prefer to administer a low titer superantigen, such as, for example SEE, instead of a high titer superantigen, such as, for example, SEB (Staphylococcal enterotoxin B). However, as has also been discovered, different superantigens have differing T-cell activation properties relative to one another, and for wild-type superantigens, the best T-cell activating superantigens often also have undesirably high seroreactivity.

These relative titers sometimes correspond to potential problems with seroreactivity, such as problems with neutralizing antibodies. Thus, the use of a low titer superantigen, such as SEA or SEE may be helpful in reducing or avoiding seroreactivity of parenterally administered superantigens. A low titer superantigen has a low seroreactivity as measured, for example, by typical anti-superantigen antibodies in a general population. In some instances it may also have a low immunogenicity. Such low titer superantigens may be modified to retain its low titer as described herein.

Approaches for modifying superantigens can be used to create superantigens that have both the desired T-cell activation properties and reduced seroreactivity, and in some instances also reduced immunogenicity. Given that certain regions of homology between superantigens relate to seroreactivity, it is possible to engineer a recombinant superantigen that has a desired T-cell activation and a desired seroreactivity and/or immunogenicity. Furthermore, the protein sequences and immunological cross-reactivity of the superantigens or staphylococcal enterotoxins are divided into two related groups. One group consists of SEA, SEE and SED. The second group is SPEA, SEC and SEB. Thus, it is possible to select low titer superantigens to decrease or eliminate the cross-reactivity with high titer or endogenous antibodies directed against staphylococcal enterotoxins.

Regions in the superantigens that are believed to play a role in seroreactivity include, for example, Region A, which comprises amino acid residues 20, 21, 22, 23, 24, 25, 26, and 27; Region B, which comprises amino acid residues 34, 35, 36, 37, 38, 39, 40, 41, 42, 43, 44, 45, 46, 47, 48, and 49; Region C, which comprises amino acid residues 74, 75, 76, 77, 78, 79, 80, 81, 82, 83, and 84; Region D, which comprises amino acid residues 187, 188, 189 and 190; and Region E, which comprise the amino acid residues, 217, 218, 219, 220, 221, 222, 223, 224, 225, 226, and 227 (see, U.S. Pat. No. 7,125,554, and FIG. 1 herein). Thus, it is contemplated that these regions can be mutated using, for example amino acid substitution, to produce a superantigen having altered seroreactivity.

Polypeptide or amino acid sequences for the above listed superantigens can be obtained from any sequence data bank, for example Protein Data Bank and/or GenBank. Exemplary GenBank accession numbers include, but are not limited to, SEE is P12993; SEA is P013163; SEB is P01552; SEC1 is P01553; SED is P20723; and SEH is AAA19777.

In certain embodiments of the present invention, the wild-type SEE sequence (SEQ ID NO: 1) or the wild type SEA sequence (SEQ ID NO: 2) can be modified such that amino acids in any of the identified regions A-E (see, FIG. 1) are substituted with other amino acids. Such substitutions include for example, K79, K81, K83 and D227 or K79, K81, K83, K84 and D227, or, for example, K79E, K81E, K83S and D227S or K79E, K81E, K83S, K84S and D227A. In certain embodiments, the superantigen is SEA/E-120 (SEQ ID NO: 3; see also U.S. Pat. No. 7,125,554) or SEA_{D227A} (SEQ ID NO: 4; see also U.S. Pat. No. 7,226,601).

1. Modified Polynucleotides and Polypeptides

A biological functional equivalent of a polynucleotide encoding a naturally occurring or a reference superantigen may comprise a polynucleotide that has been engineered to contain distinct sequences while at the same time retaining the capacity to encode the naturally occurring or reference superantigen. This can be accomplished due to the degeneracy of the genetic code, i.e., the presence of multiple codons, which encode for the same amino acids. In one example, it is possible to introduce a restriction enzyme recognition sequence into a polynucleotide while not disturbing the ability of that polynucleotide to encode a protein. Other polynucleotide sequences may encode superantigens that are different but functionally substantially equivalent in at least one biological property or activity (for example, at least 50%, 60%, 70%, 80%, 90%, 95%, 98% of the biological property or activity, for example, without limitation, the ability to induce a T-cell response that results in cytotoxicity of the tumor cells) to a reference superantigen.

In another example, a polynucleotide may be (and encode) a superantigen functionally equivalent to a reference superantigen even though it may contain more significant changes. Certain amino acids may be substituted for other amino acids in a protein structure without appreciable loss of interactive binding capacity with structures such as, for example, antigen-binding regions of antibodies, binding sites on substrate molecules, receptors, and such like. Furthermore, conservative amino acid replacements may not disrupt the biological activity of the protein, as the resultant structural change often is not one that impacts the ability of the protein to carry out its designed function. It is thus contemplated that various changes may be made in the sequence of genes and proteins disclosed herein, while still fulfilling the goals of the present invention.

Amino acid substitutions may be designed to take advantage of the relative similarity of the amino acid side-chain substituents, for example, their hydrophobicity, hydrophilicity, charge, size, and/or the like. An analysis of the size, shape and/or type of the amino acid side-chain substituents reveals that arginine, lysine and/or histidine are all positively charged residues; that alanine, glycine and/or serine are all a similar size; and/or that phenylalanine, tryptophan and/or tyrosine all have a generally similar shape. Therefore, based upon these considerations, arginine, lysine and/or histidine; alanine, glycine and/or serine; and/or phenylalanine, tryptophan and/or tyrosine; are defined herein as biologically functional equivalents. In addition, it may be possible to introduce non-naturally occurring amino acids. Approaches for making amino acid substitutions with other naturally occurring and non-naturally occurring amino acid are described in U.S. Pat. No. 7,763,253.

In terms of functional equivalents, it is understood that, implicit in the definition of a “biologically functional equivalent” protein and/or polynucleotide, is the concept that there is a limited number of changes that may be made within a defined portion of the molecule while retaining a molecule with an acceptable level of equivalent biological activity. Biologically functional equivalents are thus considered to be those proteins (and polynucleotides) where selected amino acids (or codons) may be substituted without substantially affecting biological function. Functional activity includes the induction of the T-cell response to result in cytotoxicity of the tumor cells.

In addition, it is contemplated that a modified superantigen can be created by substituting homologous regions of various proteins via “domain swapping,” which involves the generation of chimeric molecules using different but, in this case, related polypeptides. By comparing various superantigen proteins to identify functionally related regions of these molecules (see, e.g., FIG. 1), it is possible to swap related domains of these molecules so as to determine the criticality of these regions to superantigen function. These molecules may have additional value in that these “chimeras” can be distinguished from natural molecules, while possibly providing the same function.

In certain embodiments, the superantigen comprises an amino acid sequence that is at least 70%, 75%, 80%, 85%, 90%, 95%, 98%, or 99% identical to the sequence of a reference superantigen selected from SEQ ID NO: 1, SEQ ID NO: 2, SEQ ID NO: 3, and SEQ ID NO: 4, wherein the superantigen optionally retains at least 50%, 60%, 70% 80%, 90%, 95%. 98%, 99%, or 100% of a biological activity or property of the reference superantigen.

In certain embodiments, the superantigen comprises an amino acid sequence that is encoded by a nucleic acid that is at least 70%, 75%, 80%, 85%, 90%, 95%, 98%, or 99% identical to a nucleic acid encoding the superantigen selected from SEQ ID NO: 1, SEQ ID NO: 2, SEQ ID NO: 3, and SEQ ID NO: 4, wherein the superantigen optionally retains at least 50%, 60%, 70% 80%, 90%, 95%. 98%, 99%, or 100% of a biological activity or property of the reference superantigen.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EvoL. 36, 290-300; Altschul et al., (1997) NUCLEIC ACIDS RES. 25:3389-3402, incorporated by reference) are tailored for sequence similarity searching. For a discussion of basic issues in searching sequence databases see Altschul et al., (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=−3; -r, reward for nucleotide match [Integer]: default=1; -e, expect value [Real]: default=10; -W, wordsize [Integer]: default=11 for nucleotides/28 for megablast/3 for proteins; -y, Dropoff (X) for blast extensions in bits: default=20 for blastn/7 for others; -X, X dropoff value for gapped alignment (in bits): default=15 for all programs, not applicable to blastn; and -Z, final X dropoff value for gapped alignment (in bits): 50 for blastn, 25 for others). ClustalW for pairwise protein alignments may also be used (default parameters may include, e.g., Blosum62 matrix and Gap Opening Penalty =10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

### C. Targeted Superantigens

In order to increase specificity, the superantigen preferably is conjugated to a targeting moiety to create a targeted superantigen conjugate that binds an antigen preferentially expressed by a cancer cell, for example, a cell surface antigen such as 5T4. The targeting moiety is a vehicle that can be used to bind superantigen to the cancerous cells, for example, the surface of the cancerous cells. The targeted superantigen conjugate should retain the ability to activate large numbers of T lymphocytes. For example, the targeted superantigen conjugate should activate large numbers of T-cells and direct them to tissues containing the tumor-associated antigen bound to the targeting moiety. In such situations, specific target cells are preferentially killed, leaving the rest of the body relatively unharmed. This type of therapy is desirable, as non-specific anti-cancer agents, such as cytostatic chemotherapeutic drugs, are nonspecific and kill large numbers of cells not associated with tumors to be treated. For example, studies with targeted superantigen conjugates have shown that inflammation with infiltration by cytotoxic T lymphocytes (CTLs) into tumor tissue increases rapidly in response to the first injection of a targeted superantigen (Dohlsten et al. (1995) PROC. NATL. ACAD. So. USA 92:9791-9795). This inflammation with infiltration of CTLs into the tumor is one of the major effectors of the anti-tumor therapeutic of targeted superantigens.

Tumor-targeted superantigens represent an immunotherapy against cancer and are therapeutic fusion proteins containing a targeting moiety conjugated to a superantigen (Dohlsten et al. (1991) PROC. NATL. ACAD. So. USA 88:9287-9291; Dohlsten et al. (1994) PROC. NATL. ACAD. So. USA 91:8945-8949).

The targeting moiety can in principle be any structure that is able to bind to a cellular molecule, for example, a cell surface molecule and preferably is a disease specific molecule. The targeted molecule (e.g., antigen) against which the targeting moiety is directed is usually different from (a) the VP chain epitope to which superantigen binds, and (b) the MHC class II epitopes to which superantigens bind. The targeting moiety can be selected from antibodies, including antigen binding fragments thereof, soluble T-cell receptors, growth factors, interleukins (e.g., interleukin-2), hormones, etc.

In certain preferred embodiments, the targeting moiety is an antibody (e.g., Fab, F(ab)2, Fv, single chain antibody, etc.). Antibodies are extremely versatile and useful cell-specific targeting moieties because they typically can be generated against any cell surface antigen of interest. Monoclonal antibodies have been generated against cell surface receptors, tumor-associated antigens, and leukocyte lineage-specific markers such as CD antigens. Antibody variable region genes can be readily isolated from hybridoma cells by methods well known in the art. Exemplary tumor-associated antigens that can be used to produce a targeting moiety can include, but are not limited to gp100, Melan-A/MART, MAGE-A, MAGE (melanoma antigen E), MAGE-3, MAGE-4, MAGEA3, tyrosinase, TRP2, NY-ESO-1, CEA (carcinoembryonic antigen), PSA, p53, Mammaglobin-A, Survivin, MUC1 (mucin1)/DF3, metallopanstimulin-1 (MPS-1), Cytochrome P450 isoform 1B1, 90K/Mac-2 binding protein, Ep-CAM (MK-1), HSP-70, hTERT (TRT), LEA, LAGE-1/CAMEL, TAGE-1, GAGE, 5T4, gp70, SCP-1, c-myc, cyclin B1, MDM2, p62, Koc, IMP1, RCAS1, TA90, OA1, CT-7, HOM-MEL-40/SSX-2, SSX-1, SSX-4, HOM-TES-14/SCP-1, HOM-TES-85, HDAC5, MBD2, TRIP4, NY--CO-45, KNSL6, HIP1R, Seb4D, KIAA1416, IMP1, 90K/Mac-2 binding protein, MDM2, NY/ESO, EGFRvIII, IL-13Rα2, HER2, GD2, EGFR, PDL1, Mesothelin, PSMA, TGFβRDN, LMP1, GPC3, Fra, MG7, CD133, CMET, PSCA, Glypican3, ROR1, NKR-2, CD70 and LMNA.

Exemplary cancer-targeting antibodies can include, but are not limited to, anti-CD19 antibodies, anti-CD20 antibodies, anti-5T4 antibodies, anti-Ep-CAM antibodies, anti-Her-2/neu antibodies, anti-EGFR antibodies, anti-CEA antibodies, anti-prostate specific membrane antigen (PSMA) antibodies, and anti-IGF-1R antibodies. It is understood that the superantigen can be conjugated to an immunologically reactive antibody fragment such as C215Fab, 5T4Fab (see, WO8907947) or C242Fab (see, WO9301303).

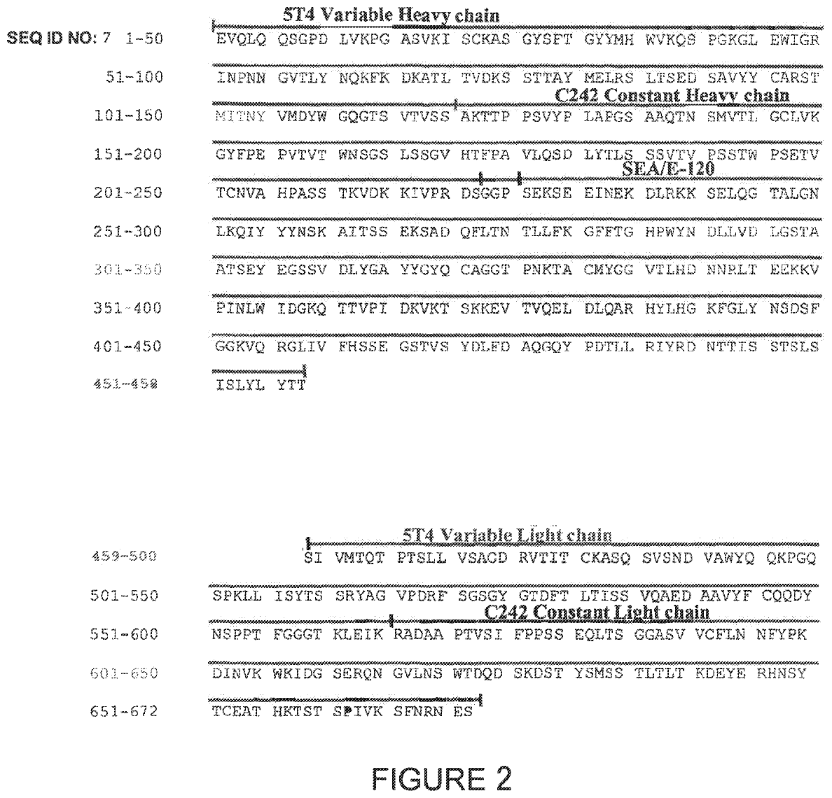

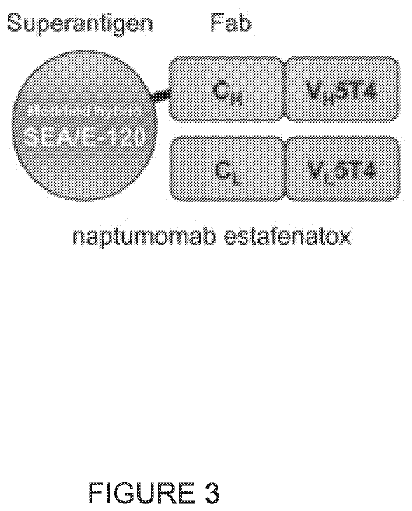

Examples of tumor targeted superantigens that can be used in the present invention include C215Fab-SEA (SEQ ID NO: 5), 5T4Fab-SEAD227A (SEQ ID NO: 6) and 5T4Fab-SEA/E-120 (SEQ ID NO: 7, see FIG. 2 and FIG. 3).

In a preferred embodiment, a preferred conjugate is a superantigen conjugate known as naptumomab estafenatox/ANYARA®, which is the fusion protein of the Fab fragment of an anti-5T4 antibody and the SEA/E-120 superantigen. Naptumomab estafenatox/ANYARA® comprises two protein chains that cumulatively include an engineered Staphylococcal enterotoxin superantigen (SEA/E-120) and a targeting 5T4 Fab comprising modified 5T4 variable region sequences fused to the constant region sequences of the murine IgG1/κ antibody C242. The first protein chain comprises residues 1 to 458 of SEQ ID NO: 7 (see also, SEQ ID NO: 8), and includes a chimeric 5T4 Fab heavy chain, corresponding to residues 1 to 222 of SEQ ID NO: 7, and the SEA/E-120 superantigen, corresponding to residues 226 to 458 of SEQ ID NO: 7, covalently linked via a GGP tripeptide linker, corresponding to residues 223-225 of SEQ ID NO: 7. The second chain comprises residues 459 to 672 of SEQ ID NO: 7 (see also, SEQ ID NO: 9) and includes a chimeric 5T4 Fab light chain. The two protein chains are held together by non-covalent interactions between the Fab heavy and light chains. Residues 1-458 of SEQ ID NO: 7 correspond to residues 1-458 of SEQ ID NO: 8, and residues 459-672 of SEQ ID NO: 7 correspond to residues 1-214 of SEQ ID NO: 9. Naptumomab estafenatox/ANYARA® comprises the proteins of SEQ ID NOS: 8 and 9 held together by non-covalent interactions between the Fab heavy and Fab light chains. Naptumomab estafenatox/ANYARA® induces T-cell mediated killing of cancer cells at concentrations around 10 pM and the superantigen component of the conjugate has been engineered to have low binding to human antibodies and MEW Class II.

It is contemplated that other antibody based targeting moieties can be designed, modified, expressed, and purified using techniques known in the art and discussed in more detail below.

Another type of targeting moiety includes a soluble T-cell receptor (TCR). Some forms of soluble TCR may contain either only extracellular domains or extracellular and cytoplasmic domains. Other modifications of the TCR may also be envisioned to produce a soluble TCR in which the transmembrane domains have been deleted and/or altered such that the TCR is not membrane bound as described in U.S. Publication Application Nos. U.S. 2002/119149, U.S. 2002/0142389, U.S. 2003/0144474, and U.S. 2003/0175212, and International Publication Nos. WO2003020763; WO9960120 and WO9960119.

The targeting moiety can be conjugated to the superantigen by using either recombinant techniques or chemically linking of the targeting moiety to the superantigen.

1. Recombinant Linker (Fusion Protein)

It is contemplated that a gene encoding a superantigen linked directly or indirectly (for example, via an amino acid containing linker) to a targeting moiety can be created and expressed using conventional recombinant DNA technologies. For example, the amino terminal of a modified superantigen can be linked to the carboxy terminal of a targeting moiety or vice versa. For antibodies, or antibody fragments that may serve as targeting moieties, either the light or the heavy chain may be utilized for creating a fusion protein. For example, for a Fab fragment, the amino terminus of the modified superantigen can be linked to the first constant domain of the heavy antibody chain (CHi). In some instances, the modified superantigen can be linked to a Fab fragment by linking the VH and VL domain to the superantigen. Alternatively, a peptide linker can be used to join the superantigen and targeting moiety together. When a linker is employed, the linker preferably contains hydrophilic amino acid residues, such as Gln, Ser, Gly, Glu, Pro, His and Arg. Preferred linkers are peptide bridges consisting of 1-10 amino acid residues, more particularly, 3-7 amino acid residues. An exemplary linker is the tripeptide-GlyGlyPro-. These approaches have been used successfully in the design and manufacture of the naptumomab estafenatox/ANYARA® superantigen conjugate.

2. Chemical Linkage

It is also contemplated that the superantigen may be linked to the targeting moiety via a chemical linkage. Chemical linkage of the superantigen to the targeting moiety may require a linker, for example, a peptide linker. The peptide linker preferably is hydrophilic and exhibits one or more reactive moieties selected from amides, thioethers, disulfides etc. (See, U.S. Pat. Nos. 5,858,363, 6,197,299, and 6,514,498). It is also contemplated that the chemical linkage can use homo- or heterobifunctional crosslinking reagents. Chemical linking of a superantigen to a targeting moiety often utilizes functional groups (e.g., primary amino groups or carboxy groups) that are present in many positions in the compounds.

### IV. Expression Methods

A protein of interest, e.g., a superantigen conjugate, a chimeric antigen receptor, and/or a T-cell receptor subunit may be expressed in a host cell of interest by incorporating a gene encoding the protein of interest into an appropriate expression vector.

Host cells can be genetically engineered, for example, by transformation or transfection technologies, to incorporate nucleic acid sequences and express the superantigen. Introduction of nucleic acid sequences into the host cell can be affected by calcium phosphate transfection, DEAE-dextran mediated transfection, microinjection, cationic lipid-mediated transfection, electroporation, transduction, scrape loading, ballistic introduction, infection or other methods. Such methods are described in many standard laboratory manuals, such as, Davis et al. (1986) BASIC METHODS IN MOLECULAR BIOLOGY and Sambrook, et al. (1989) MOLECULAR CLONING: A LABORATORY MANUAL, 2nd Ed., Cold Spring Harbor Laboratory Press, Cold Spring Harbor, N.Y.

Representative examples of appropriate host cells include bacterial cells, such as streptococci, staphylococci, E. coli, Streptomyces and Bacillus subtilis cells; fungal cells, such as yeast cells and aspergillus cells; insect cells such as Drosophila S2 and Spodoptera Sf9 cells; mammalian cells such as CHO, COS, HeLa, C127, 3T3, BHK, HEK-293 and Bowes melanoma cells.

When recombinant DNA technologies are employed a protein of interest may be expressed using standard expression vectors and expression systems. The expression vectors, which have been genetically engineered to contain the nucleic acid sequence encoding the superantigen, are introduced (e.g., transfected) into host cells to produce the superantigen (see, e.g. Dohlsten et al. (1994), Forsberg et al. (1997) J. BIOL. CHEM. 272:12430-12436, Erlandsson et al. (2003) J. MOL. BIOL. 333:893-905 and WO2003002143).

As used herein, “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes), retrotransposons (e.g. piggyback, sleeping beauty), and viruses (e.g., lentiviruses, retroviruses, adenoviruses, and adeno-associated viruses) that incorporate the recombinant polynucleotide of interest.

In certain embodiments, the expression vector is a viral vector. The term “virus” is used herein to refer to an obligate intracellular parasite having no protein-synthesizing or energy-generating mechanism. Exemplary viral vectors include retroviral vectors (e.g., lentiviral vectors), adenoviral vectors, adeno-associated viral vectors, herpesviruses vectors, epstein-barr virus (EBV) vectors, polyomavims vectors (e.g., simian vacuolating virus 40 (SV40) vectors), poxvirus vectors, and pseudotype virus vectors.

The virus may be a RNA virus (having a genome that is composed of RNA) or a DNA virus (having a genome composed of DNA). In certain embodiments, the viral vector is a DNA virus vector. Exemplary DNA viruses include parvoviruses (e.g., adeno-associated viruses), adenoviruses, asfarviruses, herpesviruses (e.g., herpes simplex virus 1 and 2 (HSV-1 and HSV-2), epstein-barr virus (EBV), cytomegalovirus (CMV)), papillornoviruses polyomaviruses (e.g., simian vacuolating virus 40 (SV40)), and poxviruses (e.g., vaccinia virus, cowpox virus, smallpox virus, fowlpox virus, sheeppox virus, myxoma virus). In certain embodiments, the viral vector is a RNA virus vector. Exemplary RNA viruses include bunyaviruses (e.g., hantavirus), coronaviruses, flaviviruses (e.g., yellow fever virus, west nile virus, dengue virus), hepatitis viruses (e.g., hepatitis A virus, hepatitis C virus, hepatitis E virus), influenza viruses (e.g., influenza virus type A, influenza virus type B, influenza virus type C), measles virus, mumps virus, noroviruses (e.g., Norwalk virus), poliovirus, respiratory syncytial virus (RSV), retroviruses (e.g., human immunodeficiency virus-1 (HIV-1)) and toroviruses.

In certain embodiments, the expression vector comprises a regulatory sequence or promoter operably linked to the nucleotide sequence encoding the protein of interest, e.g., a superantigen conjugate, a chimeric antigen receptor, and/or a T-cell receptor subunit. The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome.

Exemplary promoters which may be employed include, but are not limited to, the retroviral LTR, the SV40 promoter, the human cytomegalovirus (CMV) promoter, the U6 promoter, or any other promoter (e.g., cellular promoters such as eukaryotic cellular promoters including, but not limited to, the histone, pol III, and (3-actin promoters). Other viral promoters which may be employed include, but are not limited to, adenovirus promoters, TK promoters, and B19 parvovirus promoters.

In certain embodiments, a promoter is an inducible promoter. The use of an inducible promoter allows for expression of an operatively linked polynucleotide sequence to be turned on or off when desired. In certain embodiments, the promoter is induced in the presence of an exogenous molecule or activity, e.g., a metallothionine promoter, a glucocorticoid promoter, a progesterone promoter, and a tetracycline promoter. In certain embodiments, the promoter is induced in the tumor microenvironment, e.g., an IL-2 promoter, a NFAT promoter, a cell surface protein promoter (e.g., a CD69 promoter or a PD-1 promoter), a cytokine promoter (e.g., a TNF promoter), a cellular activation promoter (e.g., a CTLA4, OX40, or CD4OL promoter), or a cell surface adhesion protein promoter (e.g., a VLA-1 promoter).

In certain embodiments, a promoter mediates rapid, sustained expression, measured in days (e.g., a CD69 promoter). In certain embodiments, a promoter mediates delayed, late-inducible expression (e.g., a VLA1 promoter). In certain embodiments, a promoter mediates rapid, transient expression (e.g., a TNF promoter, an immediate early response gene promoter and others).

The selection of a promoter, e.g., strong, weak, inducible, tissue-specific, developmental-specific, having specific kinetics of activation (e.g., early and/or late activation), and/or having specific kinetics of expression of an induced gene (e.g., short or long expression) is within the ordinary skill of the artisan and will be apparent to those skilled in the art from the teachings contained herein.

Examples of other systems for expressing or regulating expression include “ON-Switch” CARs (Wu et al. (2015) SCIENCE 350: aab4077), combinatorial activation systems (Fedorov et al. (2014) CANCER JOURNAL 20:160-165; Kloss et al. (2013) NATURE BIOTECHNOLOGY 31: 71-75), doxycycline-inducible CARs (Sakemura et al. (2016) CANCER IMMUNOL. RES. 4:658-668), antibody-inducible CARs (Hill et al. (2018) NATURE CHEMICAL BIOLOGY 14:112-117), kill switches (Di Stasi et al. (2011) N. ENGL. J. MED. 365:1673-1683 (2011); Budde et al. (2013) PLoS ONE 8: e82742), pause switches (Wei et al. (2012) NATURE 488: 384-388), tunable receptor systems (Ma et al. (2016) PROC. NATL. ACAD. SCI. USA 113: E450-458; Rodgers et al. (2016) PROC. NATL. ACAD. SCI. USA 113: E459-468; Kudo et al. (2014) CANCER RES. 74: 93-103), and proliferation switches (Chen et al. (2010) PROC. NATL. ACAD. SCI. USA 107, 8531-8536).

Examples of production systems for superantigens are found, for example, in U.S. Pat. No. 6,962,694.

### Lentivirus Vectors

In certain embodiments, the viral vector can be a retroviral vector. Examples of retroviral vectors include moloney murine leukemia virus vectors, spleen necrosis virus vectors, and vectors derived from retroviruses such as rous sarcoma virus, harvey sarcoma virus, avian leukosis virus, human immunodeficiency virus, myeloproliferative sarcoma virus, and mammary tumor virus. Retroviral vectors are useful as agents to mediate retroviral-mediated gene transfer into eukaryotic cells.

In certain embodiments, the retroviral vector is a lentiviral vector. Exemplary lentiviral vectors include vectors derived from human immunodeficiency virus-1 (HIV-1), human immunodeficiency virus-2 (HIV-2), simian immunodeficiency virus (SIV), feline immunodeficiency virus (Hy), bovine immunodeficiency virus (BIV), Jembrana Disease Virus (JDV), equine infectious anemia virus (EIAV), and caprine arthritis encephalitis virus (CAEV).

Retroviral vectors typically are constructed such that the majority of sequences coding for the structural genes of the virus are deleted and replaced by the gene(s) of interest. Often, the structural genes (i.e., gag, pol, and env), are removed from the retroviral backbone using genetic engineering techniques known in the art. Accordingly, a minimum retroviral vector comprises from 5′ to 3′: a 5′ long terminal repeat (LTR), a packaging signal, an optional exogenous promoter and/or enhancer, an exogenous gene of interest, and a 3′ LTR. If no exogenous promoter is provided, gene expression is driven by the 5′ LTR, which is a weak promoter and requires the presence of Tat to activate expression. The structural genes can be provided in separate vectors for manufacture of the lentivirus, rendering the produced virions replication-defective. Specifically, with respect to lentivirus, the packaging system may comprise a single packaging vector encoding the Gag, Pol, Rev, and Tat genes, and a third, separate vector encoding the envelope protein Env (usually VSV-G due to its wide infectivity). To improve the safety of the packaging system, the packaging vector can be split, expressing Rev from one vector, Gag and Pol from another vector. Tat can also be eliminated from the packaging system by using a retroviral vector comprising a chimeric 5′ LTR, wherein the U3 region of the 5′ LTR is replaced with a heterologous regulatory element.

The genes can be incorporated into the proviral backbone in several general ways. The most straightforward constructions are ones in which the structural genes of the retrovirus are replaced by a single gene that is transcribed under the control of the viral regulatory sequences within the LTR. Retroviral vectors have also been constructed which can introduce more than one gene into target cells. Usually, in such vectors one gene is under the regulatory control of the viral LTR, while the second gene is expressed either off a spliced message or is under the regulation of its own, internal promoter.

Accordingly, the new gene(s) are flanked by 5′ and 3′ LTRs, which serve to promote transcription and polyadenylation of the virion RNAs, respectively. The term “long terminal repeat” or “LTR” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals, and sequences needed for replication and integration of the viral genome. The U3 region contains the enhancer and promoter elements. The U5 region is the sequence between the primer binding site and the R region and contains the polyadenylation sequence. The R (repeat) region is flanked by the U3 and U5 regions. In certain embodiments, the R region comprises a trans-activation response (TAR) genetic element, which interacts with the trans-activator (tat) genetic element to enhance viral replication. This element is not required in embodiments wherein the U3 region of the 5′ LTR is replaced by a heterologous promoter.

In certain embodiments, the retroviral vector comprises a modified 5′ LTR and/or 3′ LTR. Modifications of the 3′ LTR are often made to improve the safety of lentiviral or retroviral systems by rendering viruses replication-defective. In specific embodiments, the retroviral vector is a self-inactivating (SIN) vector. As used herein, a SIN retroviral vector refers to a replication-defective retroviral vector in which the 3′ LTR U3 region has been modified (e.g., by deletion or substitution) to prevent viral transcription beyond the first round of viral replication. This is because the 3′ LTR U3 region is used as a template for the 5′ LTR U3 region during viral replication and, thus, the viral transcript cannot be made without the U3 enhancer-promoter. In a further embodiment, the 3′ LTR is modified such that the U5 region is replaced, for example, with an ideal polyadenylation sequence. It should be noted that modifications to the LTRs such as modifications to the 3′ LTR, the 5′ LTR, or both 3′ and 5′ LTRs, are also included in the invention.

In certain embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus, because there is no complete U3 sequence in the virus production system.

Adjacent the 5′ LTR are sequences necessary for reverse transcription of the genome and for efficient packaging of viral RNA into particles (the Psi site). As used herein, the term “packaging signal” or “packaging sequence” refers to sequences located within the retroviral genome which are required for encapsidation of retroviral RNA strands during viral particle formation (see e.g., Clever et al., 1995 J. VIROLOGY, 69(4):2101-09). The packaging signal may be a minimal packaging signal (also referred to as the psi [ψ] sequence) needed for encapsidation of the viral genome.

In certain embodiments, the retroviral vector (e.g., lentiviral vector) further comprises a FLAP. As used herein, the term “FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou et al. (2000) CELL, 101:173. During reverse transcription, central initiation of the plus-strand DNA at the cPPT and central termination at the CTS lead to the formation of a three-stranded DNA structure: a central DNA flap. While not wishing to be bound by any theory, the DNA flap may act as a cis-active determinant of lentiviral genome nuclear import and/or may increase the titer of the virus. In particular embodiments, the retroviral vector backbones comprise one or more FLAP elements upstream or downstream of the heterologous genes of interest in the vectors. For example, in particular embodiments, a transfer plasmid includes a FLAP element. In one embodiment, a vector of the invention comprises a FLAP element isolated from HIV-1.

In certain embodiments, the retroviral vector (e.g., lentiviral vector) further comprises an export element. In one embodiment, retroviral vectors comprise one or more export elements. The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) RRE (see e.g., Cullen et al., (1991) J. VIROL. 65: 1053; and Cullen et al., (1991) CELL 58: 423) and the hepatitis B virus post-transcriptional regulatory element (HPRE). Generally, the RNA export element is placed within the 3′ UTR of a gene, and can be inserted as one or multiple copies.

In certain embodiments, the retroviral vector (e.g., lentiviral vector) further comprises a posttranscriptional regulatory element. A variety of posttranscriptional regulatory elements can increase expression of a heterologous nucleic acid, e.g., woodchuck hepatitis virus posttranscriptional regulatory element (WPRE; see Zufferey et al., (1999) J. VIROL., 73:2886); the posttranscriptional regulatory element present in hepatitis B virus (HPRE) (Huang et al., MOL. CELL. BIOL., 5:3864); and the like (Liu et al., (1995), GENES DEV., 9:1766). The posttranscriptional regulatory element is generally positioned at the 3′ end the heterologous nucleic acid sequence. This configuration results in synthesis of an mRNA transcript whose 5′ portion comprises the heterologous nucleic acid coding sequences and whose 3′ portion comprises the posttranscriptional regulatory element sequence. In certain embodiments, vectors of the invention lack or do not comprise a posttranscriptional regulatory element such as a WPRE or HPRE, because in some instances these elements increase the risk of cellular transformation and/or do not substantially or significantly increase the amount of mRNA transcript or increase mRNA stability. Therefore, in certain embodiments, vectors of the invention lack or do not comprise a WPRE or HPRE as an added safety measure.

Elements directing the efficient termination and polyadenylation of the heterologous nucleic acid transcripts increase heterologous gene expression. Transcription termination signals are generally found downstream of the polyadenylation signal. Accordingly, in certain embodiments, the retroviral vector (e.g., lentiviral vector) further comprises a polyadenylation signal. The term “polyadenylation signal” or “polyadenylation sequence” as used herein denotes a DNA sequence which directs both the termination and polyadenylation of the nascent RNA transcript by RNA polymerase H. Efficient polyadenylation of the recombinant transcript is desirable as transcripts lacking a polyadenylation signal are unstable and are rapidly degraded. Illustrative examples of polyadenylation signals that can be used in a vector of the invention, includes an ideal polyadenylation sequence (e.g., AATAAA, ATTAAA AGTAAA), a bovine growth hormone polyadenylation sequence (BGHpA), a rabbit β-globin polyadenylation sequence (rβgpA), or another suitable heterologous or endogenous polyadenylation sequence known in the art.

In certain embodiments, a retroviral vector further comprises an insulator element. Insulator elements may contribute to protecting retrovirus-expressed sequences, e.g., therapeutic genes, from integration site effects, which may be mediated by cis-acting elements present in genomic DNA and lead to deregulated expression of transferred sequences (i.e., position effect; see, e.g., Burgess-Beusse et al., (2002) PROC. NATL. ACAD. SCI., USA, 99:16433; and Zhan et al., 2001, Hum. GENET., 109:471). In certain embodiments, the retroviral vector comprises an insulator element in one or both LTRs or elsewhere in the region of the vector that integrates into the cellular genome. Suitable insulators for use in the invention include, but are not limited to, the chicken β-globin insulator (see Chung et al., (1993). CELL 74:505; Chung et al., (1997) PROC. NATL. ACAD. SCI., USA 94:575; and Bell et al., 1999. CELL 98:387). Examples of insulator elements include, but are not limited to, an insulator from a β-globin locus, such as chicken HS4.

Non-limiting examples of lentiviral vectors include pLVX-EFlalpha-AcGFP1-C1 (Clontech Catalog #631984), pLVX-EFlalpha-IRES-mCherry (Clontech Catalog #631987), pLVX-Puro (Clontech Catalog #632159), pLVX-IRES-Puro (Clontech Catalog #632186), pLenti6N5-DEST™ (Thermo Fisher), pLenti6.2/V5-DEST™ (Thermo Fisher), pLK0.1 (Plasmid #10878 at Addgene), pLKO.3G (Plasmid #14748 at Addgene), pSico (Plasmid #11578 at Addgene), pLJM1-EGFP (Plasmid #19319 at Addgene), FUGW (Plasmid #14883 at Addgene), pLVTHM (Plasmid #12247 at Addgene), pLVUT-tTR-KRAB (Plasmid #11651 at Addgene), pLL3.7 (Plasmid #11795 at Addgene), pLB (Plasmid #11619 at Addgene), pWPXL (Plasmid #12257 at Addgene), pWPI (Plasmid #12254 at Addgene), EF.CMV.RFP (Plasmid #17619 at Addgene), pLenti CMV Puro DEST (Plasmid #17452 at Addgene), pLenti-puro (Plasmid #39481 at Addgene), pULTRA (Plasmid #24129 at Addgene), pLX301 (Plasmid #25895 at Addgene), pHIV-EGFP (Plasmid #21373 at Addgene), pLV-mCherry (Plasmid #36084 at Addgene), pLionII (Plasmid #1730 at Addgene), pInducer10-mir-RUP-PheS (Plasmid #44011 at Addgene). These vectors can be modified to be suitable for therapeutic use. For example, a selection marker (e.g., puro, EGFP, or mCherry) can be deleted or replaced with a second exogenous gene of interest. Further examples of lentiviral vectors are disclosed in U.S. Pat. Nos. 7,629,153, 7,198,950, 8,329,462, 6,863,884, 6,682,907, 7,745,179, 7,250,299, 5,994,136, 6,287,814, 6,013,516, 6,797,512, 6,544,771, 5,834,256, 6,958,226, 6,207,455, 6,531,123, and 6,352,694, and PCT Publication No. WO2017/091786.

### Adeno-Associated Virus (AAV) Vectors

In certain embodiments, an expression vector is an adeno-associated virus (AAV) vector. AAV is a small, nonenveloped icosahedral virus of the genus Dependoparvovirus and family Parvovirus. AAV has a single-stranded linear DNA genome of approximately 4.7 kb. AAV is capable of infecting both dividing and quiescent cells of several tissue types, with different AAV serotypes exhibiting different tissue tropism.

AAV includes numerous serologically distinguishable types including serotypes AAV-1 to AAV-12, as well as more than 100 serotypes from nonhuman primates (See, e.g., Srivastava (2008) J. CELL BIOCHEM., 105(1): 17-24, and Gao et al. (2004) J. VIROL., 78(12), 6381-6388). The serotype of the AAV vector used in the present invention can be selected by a skilled person in the art based on the efficiency of delivery, tissue tropism, and immunogenicity. For example, AAV-1, AAV-2, AAV-4, AAV-5, AAV-8, and AAV-9 can be used for delivery to the central nervous system; AAV-1, AAV-8, and AAV-9 can be used for delivery to the heart; AAV-2 can be used for delivery to the kidney; AAV-7, AAV-8, and AAV-9 can be used for delivery to the liver; AAV-4, AAV-5, AAV-6, AAV-9 can be used for delivery to the lung, AAV-8 can be used for delivery to the pancreas, AAV-2, AAV-5, and AAV-8 can be used for delivery to the photoreceptor cells; AAV-1, AAV-2, AAV-4, AAV-5, and AAV-8 can be used for delivery to the retinal pigment epithelium; AAV-1, AAV-6, AAV-7, AAV-8, and AAV-9 can be used for delivery to the skeletal muscle. In certain embodiments, the AAV capsid protein comprises a sequence as disclosed in U.S. Pat. No. 7,198,951, such as, but not limited to, AAV-9 (SEQ ID NOs: 1-3 of U.S. Pat. No. 7,198,951), AAV-2 (SEQ ID NO: 4 of U.S. Patent No. 7,198,951), AAV-1 (SEQ ID NO: 5 of U.S. Pat. No. 7,198,951), AAV-3 (SEQ ID NO: 6 of U.S. Pat. No. 7,198,951), and AAV-8 (SEQ ID NO: 7 of U.S. Pat. No. 7,198,951). AAV serotypes identified from rhesus monkeys, e.g., rh.8, rh.10, rh.39, rh.43, and rh.74, are also contemplated in the instant invention. Besides the natural AAV serotypes, modified AAV capsids have been developed for improving efficiency of delivery, tissue tropism, and immunogenicity. Exemplary natural and modified AAV capsids are disclosed in U.S. Pat. Nos. 7,906,111, 9,493,788, and 7,198,951, and PCT Publication No. WO2017189964A2.

The wild-type AAV genome contains two 145 nucleotide inverted terminal repeats (ITRs), which contain signal sequences directing AAV replication, genome encapsidation and integration. In addition to the ITRs, three AAV promoters, p5, p19, and p40, drive expression of two open reading frames encoding rep and cap genes. Two rep promoters, coupled with differential splicing of the single AAV intron, result in the production of four rep proteins (Rep 78, Rep 68, Rep 52, and Rep 40) from the rep gene. Rep proteins are responsible for genomic replication. The Cap gene is expressed from the p40 promoter, and encodes three capsid proteins (VP1, VP2, and VP3) which are splice variants of the cap gene. These proteins form the capsid of the AAV particle.

Because the cis-acting signals for replication, encapsidation, and integration are contained within the ITRs, some or all of the 4.3 kb internal genome may be replaced with foreign DNA, for example, an expression cassette for an exogenous gene of interest. Accordingly, in certain embodiments, the AAV vector comprises a genome comprising an expression cassette for an exogenous gene flanked by a 5′ ITR and a 3′ ITR. The ITRs may be derived from the same serotype as the capsid or a derivative thereof. Alternatively, the ITRs may be of a different serotype from the capsid, thereby generating a pseudotyped AAV. In certain embodiments, the ITRs are derived from AAV-2. In certain embodiments, the ITRs are derived from AAV-5. At least one of the ITRs may be modified to mutate or delete the terminal resolution site, thereby allowing production of a self-complementary AAV vector.

The rep and cap proteins can be provided in trans, for example, on a plasmid, to produce an AAV vector. A host cell line permissive of AAV replication must express the rep and cap genes, the ITR-flanked expression cassette, and helper functions provided by a helper virus, for example adenoviral genes Ela, E1b55K, E2a, E4orf6, and VA (Weitzman et al., Adeno-associated virus biology. Adeno-Associated Virus: Methods and Protocols, pp. 1-23, 2011). Methods for generating and purifying AAV vectors have been described in detail (See e.g., Mueller et al., (2012) CURRENT PROTOCOLS IN MICROBIOLOGY, 14D.1.1-14D.1.21, Production and Discovery of Novel Recombinant Adeno-Associated Viral Vectors). Numerous cell types are suitable for producing AAV vectors, including HEK293 cells, COS cells, HeLa cells, BHK cells, Vero cells, as well as insect cells (See e.g. U.S. Pat. Nos. 6,156,303, 5,387,484, 5,741,683, 5,691,176, 5,688,676, and 8,163,543, U.S. Patent Publication No. 20020081721, and PCT Publication Nos. WO00/47757, WO00/24916, and WO96/17947). AAV vectors are typically produced in these cell types by one plasmid containing the ITR-flanked expression cassette, and one or more additional plasmids providing the additional AAV and helper virus genes.

AAV of any serotype may be used in the present invention. Similarly, it is contemplated that any adenoviral type may be used, and a person of skill in the art will be able to identify AAV and adenoviral types suitable for the production of their desired recombinant AAV vector (rAAV). AAV particles may be purified, for example by affinity chromatography, iodixonal gradient, or CsC1 gradient.

AAV vectors may have single-stranded genomes that are 4.7 kb in size, or are larger or smaller than 4.7 kb, including oversized genomes that are as large as 5.2 kb, or as small as 3.0 kb. Thus, where the exogenous gene of interest to be expressed from the AAV vector is small, the AAV genome may comprise a stuffer sequence. Further, vector genomes may be substantially self-complementary thereby allowing for rapid expression in the cell. In certain embodiments, the genome of a self-complementary AAV vector comprises from 5′ to 3′: a 5′ ITR; a first nucleic acid sequence comprising a promoter and/or enhancer operably linked to a coding sequence of a gene of interest; a modified ITR that does not have a functional terminal resolution site; a second nucleic acid sequence complementary or substantially complementary to the first nucleic acid sequence; and a 3′ ITR. AAV vectors containing genomes of all types are suitable for use in the method of the present invention.

Non-limiting examples of AAV vectors include pAAV-MCS (Agilent Technologies), pAAVK-EF1α-MCS (System Bio Catalog # AAV502A-1), pAAVK-EF1α-MCS1-CMV-MCS2 (System Bio Catalog # AAV503A-1), pAAV-ZsGreenl (Clontech Catalog #6231), pAAV-MCS2 (Addgene Plasmid #46954), AAV-Stuffer (Addgene Plasmid #106248), pAAVscCBPIGpluc (Addgene Plasmid #35645), AAVS1_Puro_PGK1 3xFLAG_Twin_Strep (Addgene Plasmid #68375), pAAV-RAM-d2TTA::TRE-MCS-WPRE-pA (Addgene Plasmid #63931), pAAV-UbC (Addgene Plasmid #62806), pAAVS1-P-MCS (Addgene Plasmid #80488), pAAV-Gateway (Addgene Plasmid #32671), pAAV-Puro_siKD (Addgene Plasmid #86695), pAAVS1-Nst-MCS (Addgene Plasmid #80487), pAAVS1-Nst-CAG-DEST (Addgene Plasmid #80489), pAAVS1-P-CAG-DEST (Addgene Plasmid #80490), pAAVf-EnhCB-lacZnls (Addgene Plasmid #35642), and pAAVS1-shRNA (Addgene Plasmid #82697). These vectors can be modified to be suitable for therapeutic use. For example, an exogenous gene of interest can be inserted in a multiple cloning site, and a selection marker (e.g., puro or a gene encoding a fluorescent protein) can be deleted or replaced with another (same or different) exogenous gene of interest. Further examples of AAV vectors are disclosed in U.S. Pat. Nos. 5,871,982, 6,270,996, 7,238,526, 6,943,019, 6,953,690, 9,150,882, and 8,298,818, U.S. Patent Publication No. 2009/0087413, and PCT Publication Nos. WO2017075335A1, WO2017075338A2, and

### Adenoviral Vectors

In certain embodiments, the viral vector can be an adenoviral vector. Adenoviruses are medium-sized (90-100 nm), non-enveloped (naked), icosahedral viruses composed of a nucleocapsid and a double-stranded linear DNA genome. The term “adenovirus” refers to any virus in the genus Adenoviridiae including, but not limited to, human, bovine, ovine, equine, canine, porcine, murine, and simian adenovirus subgenera. Typically, an adenoviral vector is generated by introducing one or more mutations (e.g., a deletion, insertion, or substitution) into the adenoviral genome of the adenovirus so as to accommodate the insertion of a non-native nucleic acid sequence, for example, for gene transfer, into the adenovirus.

A human adenovirus can be used as the source of the adenoviral genome for the adenoviral vector. For instance, an adenovirus can be of subgroup A (e.g., serotypes 12, 18, and 31), subgroup B (e.g., serotypes 3, 7, 1 1 , 14, 16, 21 , 34, 35, and 50), subgroup C (e.g., serotypes 1 , 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serogroup or serotype. Adenoviral serotypes 1 through 51 are available from the American Type Culture Collection (ATCC, Manassas, Virginia). Non-group C adenoviral vectors, methods of producing non-group C adenoviral vectors, and methods of using non-group C adenoviral vectors are disclosed in, for example, U.S. Pat. Nos. 5,801,030, 5,837,511, and 5,849,561, and PCT Publication Nos. WO1997/012986 and WO1998/053087.

Non-human adenovirus (e.g., ape, simian, avian, canine, ovine, or bovine adenoviruses) can be used to generate the adenoviral vector (i.e., as a source of the adenoviral genome for the adenoviral vector). For example, the adenoviral vector can be based on a simian adenovirus, including both new world and old world monkeys (see, e.g., Virus Taxonomy: VHIth Report of the International Committee on Taxonomy of Viruses (2005)). A phylogeny analysis of adenoviruses that infect primates is disclosed in, e.g., Roy et al. (2009) PLoS PATHOG. 5(7):e1000503. A gorilla adenovirus can be used as the source of the adenoviral genome for the adenoviral vector. Gorilla adenoviruses and adenoviral vectors are described in, e.g., PCT Publication Nos.WO2013/052799, WO2013/052811, and WO2013/052832. The adenoviral vector can also comprise a combination of subtypes and thereby be a “chimeric” adenoviral vector.

The adenoviral vector can be replication-competent, conditionally replication-competent, or replication-deficient. A replication-competent adenoviral vector can replicate in typical host cells, i.e., cells typically capable of being infected by an adenovirus. A conditionally-replicating adenoviral vector is an adenoviral vector that has been engineered to replicate under pre-determined conditions. For example, replication-essential gene functions, e.g., gene functions encoded by the adenoviral early regions, can be operably linked to an inducible, repressible, or tissue-specific transcription control sequence, e.g., a promoter. Conditionally-replicating adenoviral vectors are further described in U.S. Pat. No. 5,998,205. A replication-deficient adenoviral vector is an adenoviral vector that requires complementation of one or more gene functions or regions of the adenoviral genome that are required for replication, as a result of, for example, a deficiency in one or more replication-essential gene function or regions, such that the adenoviral vector does not replicate in typical host cells, especially those in a human to be infected by the adenoviral vector.

Preferably, the adenoviral vector is replication-deficient, such that the replication-deficient adenoviral vector requires complementation of at least one replication-essential gene function of one or more regions of the adenoviral genome for propagation (e.g., to form adenoviral vector particles). The adenoviral vector can be deficient in one or more replication-essential gene functions of only the early regions (i.e., E1-E4 regions) of the adenoviral genome, only the late regions (i.e., L1-L5 regions) of the adenoviral genome, both the early and late regions of the adenoviral genome, or all adenoviral genes (i.e., a high capacity adenovector (HC-Ad)). See, e.g., Morsy et al. (1998) PROC. NATL. ACAD. SCI. USA 95: 965-976, Chen et al. (1997) PROC. NATL. ACAD. SCI. USA 94: 1645-1650, and Kochanek et al. (1999) HUM. GENE THER. 10(15):2451-9. Examples of replication-deficient adenoviral vectors are disclosed in U.S. Pat. Nos. 5,837,511, 5,851,806, 5,994,106, 6,127,175, 6,482,616, and 7,195,896, and PCT Publication Nos. WO1994/028152, WO1995/002697, WO1995/016772, WO1995/034671, WO1996/022378, WO1997/012986, WO1997/021826, and WO2003/022311.

The replication-deficient adenoviral vector of the invention can be produced in complementing cell lines that provide gene functions not present in the replication-deficient adenoviral vector, but required for viral propagation, at appropriate levels in order to generate high titers of viral vector stock. Such complementing cell lines are known and include, but are not limited to, 293 cells (described in, e.g., Graham et al. (1977) J. GEN. VIROL. 36: 59-72), PER.C6 cells (described in, e.g., PCT Publication No. WO1997/000326, and U.S. Pat. Nos. 5,994,128 and 6,033,908), and 293-ORF6 cells (described in, e.g., PCT Publication No. WO1995/034671 and Brough et al. (1997) J. VIROL. 71: 9206-9213). Other suitable complementing cell lines to produce the replication-deficient adenoviral vector of the invention include complementing cells that have been generated to propagate adenoviral vectors encoding transgenes whose expression inhibits viral growth in host cells (see, e.g., U.S. Patent Publication No. 2008/0233650). Additional suitable complementing cells are described in, for example, U.S. Pat. Nos. 6,677,156 and 6,682,929, and PCT Publication No. WO2003/020879. Formulations for adenoviral vector-containing compositions are further described in, for example, U.S. Pat. Nos. 6,225,289, and 6,514,943, and PCT Publication No. WO2000/034444.

Additional exemplary adenoviral vectors, and/or methods for making or propagating adenoviral vectors are described in U.S. Pat. Nos. 5,559,099, 5,837,511, 5,846,782, 5,851,806, 5,994,106, 5,994,128, 5,965,541, 5,981,225, 6,040,174, 6,020,191, 6,083,716, 6,113,913, 6,303,362, 7,067,310, and 9,073,980.

Commercially available adenoviral vector systems include the ViraPower™ Adenoviral Expression System available from Thermo Fisher Scientific, the AdEasy™ adenoviral vector system available from Agilent Technologies, and the Adeno-X™ Expression System 3 available from Takara Bio USA, Inc.

### Viral Vector Production

Methods for producing viral vectors are known in the art. Typically, a virus of interest is produced in a suitable host cell line using conventional techniques including culturing a transfected or infected host cell under suitable conditions so as to allow the production of infectious viral particles. Nucleic acids encoding viral genes and/or genes of interest can be incorporated into plasmids and introduced into host cells through conventional transfection or transformation techniques. Exemplary suitable host cells for production of disclosed viruses include human cell lines such as HeLa, Hela-S3, HEK293, 911, A549, HER96, or PER-C6 cells. Specific production and purification conditions will vary depending upon the virus and the production system employed.

In certain embodiments, producer cells may be directly administered to a subject, however, in other embodiments, following production, infectious viral particles are recovered from the culture and optionally purified. Typical purification steps may include plaque purification, centrifugation, e.g., cesium chloride gradient centrifugation, clarification, enzymatic treatment, e.g., benzonase or protease treatment, chromatographic steps, e.g., ion exchange chromatography or filtration steps.

### Protein Purification

The superantigen and/or the superantigen-targeting moiety conjugates preferably are purified prior to use, which can be accomplished using a variety of purification protocols. Having separated the superantigen or the superantigen-targeting moiety conjugate from other proteins, the protein of interest may be further purified using chromatographic and electrophoretic techniques to achieve partial or complete purification (or purification to homogeneity). Analytical methods particularly suited to the preparation of a pure peptide are ion-exchange chromatography, size exclusion chromatography; affinity chromatography; polyacrylamide gel electrophoresis; isoelectric focusing. The term “purified” as used herein, is intended to refer to a composition, isolatable from other components, wherein the macromolecule (e.g., protein) of interest is purified to any degree relative to its original state. Generally, the terms “purified” refer to a macromolecule that has been subjected to fractionation to remove various other components, and which substantially retains its biological activity. The term “substantially purified” refers to a composition in which the macromolecule of interest forms the major component of the composition, such as constituting about 50%, about 60%, about 70%, about 80%, about 90%, about 95% or more of the content of the composition.

Various methods for quantifying the degree of purification of the protein are known to those of skill in the art, including, for example, determining the specific activity of an active fraction, and assessing the amount of a given protein within a fraction by SDS-PAGE analysis, High Performance Liquid Chromatography (HPLC), or any other fractionation technique. Various techniques suitable for use in protein purification include, for example, precipitation with ammonium sulfate, PEG, antibodies and the like or by heat denaturation, followed by centrifugation; chromatography steps such as ion exchange, gel filtration, reverse phase, hydroxyapatite, affinity chromatography; isoelectric focusing; gel electrophoresis; and combinations of such and other techniques. It is contemplated that the order of conducting the various purification steps may be changed, or that certain steps may be omitted, and still result in a suitable method for the preparation of a substantially purified protein or peptide.

### V. Pharmaceutical Compositions

For therapeutic use, an immune cell (for example, an isolated naturally occurring immune cell or an engineered immune cell described herein) and/or a superantigen conjugate preferably is combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (See Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) BIOENG. TRANSL. MED. 1: 10-29).

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(-)-3-hydroxybutyric acid. Sustained release compositions may also include liposomes that can be prepared by any of several methods known in the art.

Pharmaceutical compositions containing an immune cell and/or a superantigen conjugate disclosed herein can be presented in a dosage unit form and can be prepared by any suitable method. A pharmaceutical composition should be formulated to be compatible with its intended route of administration. Examples of routes of administration are intravenous (IV), intramuscular, intradermal, inhalation, transdermal, topical, transmucosal, intrathecal and rectal administration. In certain embodiments, a pharmaceutical composition containing an immune cell and/or a a superantigen conjugate disclosed herein is administered by IV infusion. Alternatively, the agents may be administered locally rather than systemically, for example, via injection of the agent or agents directly into the site of action, often in a depot or sustained release formulation. In certain embodiments, a pharmaceutical composition containing an immune cell and/or a a superantigen conjugate disclosed herein is administered by intratumoral injection.

Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution.

In certain embodiments, pharmaceutical compositions may comprise, for example, at least about 0.1% of an active compound. In other embodiments, the active compound may comprise between about 2% to about 75% of the weight of the unit, or between about 25% to about 60%, for example, and any range derivable therein. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable. Such determinations are known and used by those of skill in the art.

The active agents are administered in an amount or amounts effective to decrease, reduce, inhibit or otherwise abrogate the growth or proliferation of cancer cells, induce apoptosis, inhibit angiogenesis of a cancer or tumor, inhibit metastasis, or induce cytotoxicity in cells. The effective amount of active compound(s) used to practice the present invention for therapeutic treatment of cancer varies depending upon the manner of administration, the age, body weight, and general health of the subject. These terms include synergistic situations wherein a single agent alone, such as a superantigen conjugate or an immune cell may act weakly or not at all, but when combined with each other, for example, but not limited to, via sequential dosage, the two or more agents act to produce a synergistic result.

Generally, a therapeutically effective amount of active component is in the range of 0.1 mg/kg to 100 mg/kg, e.g., 1 mg/kg to 100 mg/kg, 1 mg/kg to 10 mg/kg. The amount administered will depend on variables such as the type and extent of disease or indication to be treated, the overall health of the patient, the in vivo potency of the antibody, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the antibody, and the disease being treated. Exemplary dosing frequencies are once per day, once per week and once every two weeks. A preferred route of administration is parenteral, e.g., intravenous infusion. In certain embodiments, a superantigen conjugate is lyophilized, and then reconstituted in buffered saline, at the time of administration.

In certain non-limiting examples, a dose of isolated, naturally occurring or engineered immune cells, e.g., T-cells, is in the range of, e.g., 10⁵ to 10⁹ cells/kg, 10⁵ to 10⁸ cells/kg, 10⁵ to 10⁷ cells/kg, 10⁵ to 10⁶ cells/kg, 10⁶ to 10⁹ cells/kg, 10⁶ to 10⁸ cells/kg, 10⁶ to 10⁷ cells/kg, 10⁷ to 10⁹ cells/kg, 10⁷ to 10⁸ cells/kg, or 10⁸ to 10⁹ cells/kg, or 10⁶ to 10¹¹ total cells, 10⁶ to 10¹⁰ total cells, 10⁶ to 10⁹ total cells, 10⁶ to 10⁸ total cells, 10⁶ to 10⁷ total cells, 10⁷ to 10¹¹ total cells, 10⁷ to 10¹⁰ total cells, 10⁷ to 10⁹ total cells, 10⁷ to 10⁸ total cells, 10⁸ to 10¹¹ total cells, 10⁸ to 10¹⁰ total cells 10⁸ to 10⁹ total cells, 10⁹ to 10¹¹ total cells, 10⁹ to 10¹⁰ total cells, or 10¹⁰ to 10¹¹ total cells. The amount administered will depend on variables such as the type and extent of disease or indication to be treated, the overall health of the patient, the in vivo potency of the antibody, the pharmaceutical formulation, and the route of administration. Progress can be monitored by periodic assessment.

In certain non-limiting examples, a dose of the superantigen conjugate may also comprise from about 1 microgram/kg/body weight, about 5 microgram/kg/body weight, about 10 microgram/kg/body weight, about 15 microgram/kg/body weight, about 20 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, about 1 microgram/kg/body weight to about 100 milligram/kg/body weight. Other exemplary dosage ranges, range from about 1 microgram/kg/body weight to about 1000 microgram/kg/body weight, from about 1 microgram/kg/body weight to about 100 microgram/kg/body weight, from about 1 microgram/kg/body weight to about 75 microgram/kg/body weight, from about 1 microgram/kg/body weight to about 50 microgram/kg/body weight, from about 1 microgram/kg/body weight to about 40 microgram/kg/body weight, from about 1 microgram/kg/body weight to about 30 microgram/kg/body weight, from about 1 microgram/kg/body weight to about 20 microgram/kg/body weight, from about 1 microgram/kg/body weight to about 15 microgram/kg/body weight, from about 1 microgram/kg/body weight to about 10 microgram/kg/body weight, from about 5 microgram/kg/body weight to about 1000 microgram/kg/body weight, from about 5 microgram/kg/body weight to about 100 microgram/kg/body weight, from about 5 microgram/kg/body weight to about 75 microgram/kg/body weight, from about 5 microgram/kg/body weight to about 50 microgram/kg/body weight, from about 5 microgram/kg/body weight to about 40 microgram/kg/body weight, from about 5 microgram/kg/body weight to about 30 microgram/kg/body weight, from about 5 microgram/kg/body weight to about 20 microgram/kg/body weight, from about 5 microgram/kg/body weight to about 15 microgram/kg/body weight, from about 5 microgram/kg/body weight to about 10 microgram/kg/body weight, from about 10 microgram/kg/body weight to about 1000 microgram/kg/body weight, from about 10 microgram/kg/body weight to about 100 microgram/kg/body weight, from about 10 microgram/kg/body weight to about 75 microgram/kg/body weight, from about 10 microgram/kg/body weight to about 50 microgram/kg/body weight, from about 10 microgram/kg/body weight to about 40 microgram/kg/body weight, from about 10 microgram/kg/body weight to about 30 microgram/kg/body weight, from about 10 microgram/kg/body weight to about 20 microgram/kg/body weight, from about 10 microgram/kg/body weight to about 15 microgram/kg/body weight, from about 15 microgram/kg/body weight to about 1000 microgram/kg/body weight, from about 15 microgram/kg/body weight to about 100 microgram/kg/body weight, from about 15 microgram/kg/body weight to about 75 microgram/kg/body weight, from about 15 microgram/kg/body weight to about 50 microgram/kg/body weight, from about 15 microgram/kg/body weight to about 40 microgram/kg/body weight, from about 15 microgram/kg/body weight to about 30 microgram/kg/body weight, from about 15 microgram/kg/body weight to about 20 microgram/kg/body weight, from about 20 microgram/kg/body weight to about 1000 microgram/kg/body weight, from about 20 microgram/kg/body weight to about 100 microgram/kg/body weight, from about 20 microgram/kg/body weight to about 75 microgram/kg/body weight, from about 20 microgram/kg/body weight to about 50 microgram/kg/body weight, from about 20 microgram/kg/body weight to about 40 microgram/kg/body weight, from about 20 microgram/kg/body weight to about 30 microgram/kg/body weight, etc., can be administered, based on the numbers described above.

In certain embodiments, for example, administration of the superantigen conjugate, the effective amount or dose of the superantigen conjugate that is administered is an amount in the range of 0.01 to 500 μg/kg body weight of the subject, for example, 0.1-500 μg/kg body weight of the subject, and, for example, 1-100 μg/kg body weight of the subject.

The compositions described herein may be administered locally or systemically. Administration will generally be parenteral administration. In a preferred embodiment, the pharmaceutical composition is administered subcutaneously and in an even more preferred embodiment intravenously. Preparations for parenteral administration include sterile aqueous or non-aqueous solutions, suspensions, and emulsions.

### VI. Therapeutic Uses

The compositions and methods disclosed herein can be used to treat various forms of cancer in a subject or inhibit cancer growth in a subject. The invention provides a method of treating a cancer in a subject. The method comprises administering to the subject an effective amount of a disclosed immune cell and/or superantigen conjugate, either alone or in a combination with another therapeutic agent to treat the cancer in the subject. For example, the disclosed immune cell and/or superantigen conjugate can be administered to the subject to slow the growth rate of cancer cells, reduce the incidence or number of metastases, reduce tumor size, inhibit tumor growth, reduce the blood supply to a tumor or cancer cells, promote an immune response against cancer cells or a tumor, prevent or inhibit the progression of cancer, for example, by at least 40%, 50%, 60%, 70%, 80%, 90%, 95%, 98%, 99%, or 100%. Alternatively, the immune cell and/or superantigen conjugate can be administered to the subject so as to treat the cancer, for example, to increase the lifespan of a subject with cancer, for example, by 3 months, 6 months, 9 months, 12 months, 1 year, 5 years, or 10 years.

Preferably, patients to be treated will have adequate bone marrow function (defined as a peripheral absolute granulocyte count of >2,000/mm³ and a platelet count of 100,000/mm³), adequate liver function (bilirubin<1.5 mg/dl) and adequate renal function (creatinine<1.5 mg/dl).

It is contemplated that a number of cancers may be treated using the methods and compositions described herein, including but not limited to primary or metastatic melanoma, adenocarcinoma, squamous cell carcinoma, adenosquamous cell carcinoma, thymoma, lymphoma, sarcoma, lung cancer, liver cancer, non-Hodgkin's lymphoma, Hodgkin's lymphoma, leukemia, uterine cancer, breast cancer, prostate cancer, ovarian cancer, pancreatic cancer, colon cancer, multiple myeloma, neuroblastoma, NPC, bladder cancer, cervical cancer and the like.

Moreover, the cancer that may be treated using the methods and compositions described herein may be based upon the body location and/or system to be treated, for example, but not limited to bone (e.g., Ewing's Family of tumors, osteosarcoma); brain (e.g., adult brain tumor, (e.g., adult brain tumor, brain stem glioma (childhood), cerebellar astrocytoma (childhood), cerebral astrocytoma/malignant glioma (childhood), ependymoma (childhood). medulloblastoma (childhood), supratentorial primitive neuroectodermal tumors and pineoblastoma (childhood), visual pathway and hypothalamic glioma (childhood) and childhood brain tumor (other)); breast (e.g., female or male breast cancer); digestive/gastrointestinal (e.g., anal cancer, bile duct cancer (extrahepatic), carcinoid tumor (gastrointestinal), colon cancer, esophageal cancer, gallbladder cancer, liver cancer (adult primary), liver cancer (childhood), pancreatic cancer, small intestine cancer, stomach (gastric) cancer); endocrine (e.g., adrenocortical carcinoma, carcinoid tumor (gastrointestinal), islet cell carcinoma (endocrine pancreas), parathyroid cancer, pheochromocytoma, pituitary tumor, thyroid cancer); eye (e.g., melanoma (intraocular), retinoblastoma); genitourinary (e.g., bladder cancer, kidney (renal cell) cancer, penile cancer, prostate cancer, renal pelvis and ureter cancer (transitional cell), testicular cancer, urethral cancer, Wilms' Tumor and other childhood kidney tumors); germ cell (e.g., extracranial germ cell tumor (childhood), extragonadal germ cell tumor, ovarian germ cell tumor, testicular cancer); gynecologic (e.g., cervical cancer, endometrial cancer, gestational trophoblastic tumor, ovarian epithelial cancer, ovarian germ cell tumor, ovarian low malignant potential tumor, uterine sarcoma, vaginal cancer, vulvar cancer); head and neck (e.g., hypopharyngeal cancer, laryngeal cancer, lip and oral cavity cancer, metastatic squamous neck cancer with occult primary, nasopharyngeal cancer, oropharyngeal cancer, paranasal sinus and nasal cavity cancer, parathyroid cancer, salivary gland cancer); lung (e.g., non-small cell lung cancer, small cell lung cancer); lymphoma (e.g., AIDS-Related Lymphoma, cutaneous T-cell lymphoma, Hodgkin's Lymphoma (adult), Hodgkin's Lymphoma (childhood), Hodgkin's Lymphoma during pregnancy, mycosis fungoides, Non-Hodgkin's Lymphoma (adult), Non-Hodgkin's Lymphoma (childhood), Non-Hodgkin's Lymphoma during pregnancy, primary central nervous system lymphoma, Sezary Syndrome, T-cell lymphoma (cutaneous), Waldenstrom's Macroglobulinemia); musculoskeletal (e.g., Ewing's Family of tumors, osteosarcoma/malignant fibrous histiocytoma of bone, rhabdomyosarcoma (childhood), soft tissue sarcoma (adult), soft tissue sarcoma (childhood), uterine sarcoma); neurologic (e.g., adult brain tumor, childhood brain tumor (e.g., brain stem glioma, cerebellar astrocytoma, cerebral astrocytoma/malignant glioma, ependymoma, medulloblastoma, supratentorial primitive neuroectodermal tumors and pineoblastoma, visual pathway and hypothalamic glioma, other brain tumors), neuroblastoma, pituitary tumor primary central nervous system lymphoma); respiratory/thoracic (e.g., non-small cell lung cancer, small cell lung cancer, malignant mesothelioma, thymoma and thymic carcinoma); and skin (e.g., cutaneous T-cell lymphoma, Kaposi's sarcoma, melanoma, and skin cancer).

It is understood that the method can be used to treat a variety of cancers, for example, a cancer selected from breast cancer, bladder cancer, cervical cancer, colon cancer, colorectal cancer, endometrial cancer, gastric cancer, head and neck cancer, liver cancer, melanoma, mesothelioma, non-small cell lung cancer, ovarian cancer, pancreatic cancer, prostate cancer, renal cell cancer, and skin cancer.

Yet further, the cancer may include a tumor comprised of tumor cells. For example, tumor cells may include, but are not limited to melanoma cell, a bladder cancer cell, a breast cancer cell, a lung cancer cell, a colon cancer cell, a prostate cancer cell, a liver cancer cell, a pancreatic cancer cell, a stomach cancer cell, a testicular cancer cell, a renal cancer cell, an ovarian cancer cell, a lymphatic cancer cell, a skin cancer cell, a brain cancer cell, a bone cancer cell, or a soft tissue cancer cell. Examples of solid tumors that can be treated according to the invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Treatment regimens may vary as well, and often depend on tumor type, tumor location, disease progression, and health and age of the patient. Certain types of tumor may require more aggressive treatment protocols, but at the same time, the patients may be unable to tolerate more aggressive treatment regimens. The clinician may often be best suited to make such decisions based on his or her skill in the art and the known efficacy and toxicity (if any) of the therapeutic formulations.

A typical course of treatment, for a primary tumor or a post-excision tumor bed, may involve multiple doses. Typical primary tumor treatment may involve a 6 dose application over a two-week period. The two-week regimen may be repeated one, two, three, four, five, six or more times. During a course of treatment, the need to complete the planned dosings may be re-evaluated.

Immunotherapy with the superantigen conjugate often results in rapid (within hours) and powerful polyclonal activation of T lymphocytes. A superantigen conjugate treatment cycle may include 4 to 5 daily intravenous superantigen conjugate drug injections. Such treatment cycles can be given in e.g., 4 to 6 week intervals. The inflammation with infiltration of CTLs into the tumor is one of the major effectors of the anti-tumor therapeutic superantigens. After a short period of massive activation and differentiation of CTLs, the T-cell response declines rapidly (within 4-5 days) back to base line levels. Thus, the period of lymphocyte proliferation, during which cytostatic drugs may interfere with superantigen treatment is short and well-defined.

In certain embodiments, a subject is administered a superantigen conjugate, e.g., a superantigen conjugate contemplated herein, daily for 2 to 6 consecutive days (e.g., 2, 3, 4, 5, or 6 consecutive days) every 2 to 12 weeks (e.g., 2, 3, 4, 5, 6, 7, 8, 9, 10, 11, or 12 weeks). In certain embodiments, a subject is administered a superantigen conjugate, e.g., a superantigen conjugate contemplated herein, daily for 4 consecutive days every 3 to 4 weeks (e.g., 3 or 4 weeks).

In certain embodiments, the treatment regimen of the present invention may involve contacting the neoplasm or tumor cells with the superantigen conjugate and the immune cell, e.g., CAR T-cell, at the same time. This may be achieved by contacting the cell with a single composition or pharmacological formulation that includes both agents, or by contacting the cell with two distinct compositions or formulations, at the same time, wherein one composition includes the superantigen conjugate and the other includes the immune cell, e.g., CAR T-cell.

Alternatively, the superantigen conjugate may precede or follow the immune cell, e.g., CAR T-cell, by intervals ranging from minutes, days to weeks. In embodiments where the immune cell, e.g., CAR T-cell, and the superantigen conjugate are applied separately to the cell, one should ensure that a significant period of time does not expire between the time of each delivery, such that the superantigen conjugate and immune cell, e.g., CAR T-cell, would still be able to exert an advantageously combined effect on the cell. In such instances, it is contemplated that one may contact the cell with both modalities within about 12-72 hours of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, the superantigen conjugate being “A” and the immune cell, e.g., CAR T-cell, being “B”: AB/A, B/A/B, BB/A, A/A/B, A/B/B, B/A/A, A/BBB, B/A/B/B, BBB/A, B/B/A/B, A/A/B/B, AB/AB, A/B/B/A, B/B/A/A, B/A/B/A, B/A/A/B, A/A/A/B, B/A/A/A, A/B/A/A, and A/A/B/A.

It is envisioned that the effective amount or dose of immune cell, e.g., CAR T-cell, that is administered in combination with the superantigen conjugate is a dose that results in an at least an additive but preferably a synergistic anti-tumor effect and does not interfere or inhibit the enhancement of the immune system or T-cell activation. If the immune cell, e.g., CAR T-cell, is administered simultaneously with the superantigen conjugate, then the immune cell, e.g., CAR T-cell, may be administered in a low dose such that it does not interfere with the mechanism of action of the superantigen conjugate.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a method or composition described herein, is administered in combination with one or more additional therapies, e.g., surgery, radiation therapy, or administration of another therapeutic preparation. In certain embodiments, the additional therapy may include chemotherapy, e.g., a cytotoxic agent. In certain embodiments the additional therapy may include a targeted therapy, e.g. a tyrosine kinase inhibitor, a proteasome inhibitor, or a protease inhibitor. In certain embodiments, the additional therapy may include an anti-inflammatory, anti-angiogenic, anti-fibrotic, or anti-proliferative compound, e.g., a steroid, a biologic immunomodulator, a monoclonal antibody, an antibody fragment, an aptamer, an siRNA, an antisense molecule, a fusion protein, a cytokine, a cytokine receptor, a bronchodialator, a statin, an anti-inflammatory agent (e.g. methotrexate), or an NSAID. In certain embodiments, the additional therapy may include a compound designed to reduce the subject's possible immunoreactivity to the administered superantigen conjugate. For example, immunoreactivity to the administered superantigen may be reduced via co-administration with, for example, an anti-CD20 antibody and/or an anti-CD19 antibody, that reduces the production of anti-superantigen antibodies in the subject. In certain embodiments, the additional therapy may include a combination of therapeutics of different classes.

In certain embodiments, a method or composition described herein is administered in combination with an immunopotentiator.

In certain embodiments, exemplary immunopotentiators can: (a) stimulate activating T-cell signaling, (b) repress T-cell inhibitory signalling between the cancerous cells and a T-cell, (c) repress inhibitory signalling that leads to T-cell expansion, activation and/or activity via a non-human IgG1-mediated immune response pathway, for example, a human IgG4 immunoglobulin-mediated pathway, (d) a combination of (a) and (b), (e) combination of (a) and (c), (f) a combination of (b) and (c), and (g) a combination of (a), (b), and (c).

In certain embodiments, the immunopotentiator is a checkpoint pathway inhibitor. The checkpoint inhibitor may, for example, be selected from a PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist or TIGIT antagonist.

PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, PD-L2, etc., that interact with PD-1 on the surface of T-cells to shut down or modulate T-cell activity. Using this approach, cancer can evade the T-cell mediated immune response.

In the CTLA-4 pathway, the interaction of CTLA-4 on the T-cell with its ligands (e.g., CD80, also known as B7-1, and CD86) on the surface of an antigen presenting cells (rather than the cancer calls) leads to T-cell inhibition. As a result, the ligand that inhibits T-cell activation or activity (e.g., CD80 or CD86) is provided by an antigen presenting cell (a key cell type in the immune system) rather than the cancer cell. Although CTLA-4 and PD-1 binding both have similar negative effects on T-cells the timing of downregulation, the responsible signaling mechanisms, and the anatomic locations of immune inhibition by these two immune checkpoints differ (American Journal of Clinical Oncology. Volume 39, Number 1, February 2016). Unlike CTLA-4, which is confined to the early priming phase of T-cell activation, PD-1 functions much later during the effector phase, (Keir et al. (2008) ANNU. REV IMMUNOL., 26:677-704). CTLA-4 and PD-1 represent two T-cell-inhibitory receptors with independent, non-redundant mechanisms of action.

In certain embodiments, the immunopotentiator prevents (completely or partially) an antigen expressed by the cancerous cell from repressing T-cell inhibitory signaling between the cancerous cell and the T-cell. In one embodiment, such an immunopotentiator is a checkpoint inhibitor, for example, a PD-1-based inhibitor. Examples of such immunopotentiators include, for example, anti-PD-1 antibodies, anti-PD-L1 antibodies, and anti-PD-L2 antibodies.

In certain embodiments, the superantigen conjugate is administered with a PD-1-based inhibitor. A PD-1-based inhibitor can include (i) a PD-1 inhibitor, i.e., a molecule (for example, an antibody or small molecule) that binds to PD-1 on a T-cell to prevent the binding of a PD-1 ligand expressed by the cancer cell of interest, and/or (ii) a PD-L inhibitor, e.g., a PD-L1 or PD-L2 inhibitor, i.e., a molecule (for example, an antibody or small molecule) that binds to a PD-1 ligand (for example, PD-L1 or PD-L2) to prevent the PD-1 ligand from binding to its cognate PD-1 on the T-cell.

In certain embodiments the superantigen conjugate is administered with a CTLA-4 inhibitor, e.g., an anti-CTLA-4 antibody. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 B1. Exemplary CTLA-4 antibodies include ipilimumab or tremelimumab.

In certain embodiments, the immunopotentiator prevents (completely or partially) an antigen expressed by the cancerous cell from repressing T-cell expansion, activation and/or activity via a human IgG4 (a non-human IgG1) mediated immune response pathway, for example, not via an ADCC pathway. It is contemplated that, in such embodiments, although the immune response potentiated by the superantigen conjugate and the immunopotentiator may include some ADCC activity, the principal component(s) of the immune response do not involve ADCC activity. In contrast, some of the antibodies currently being used in immunotherapy, such as ipilimumab (an anti-CTLA-4 IgG1 monoclonal antibody), can kill targeted cells via ADCC through signaling via their Fc domain through Fc receptors on effector cells. Ipilimumab, like many other therapeutic antibodies, was designed as a human IgG1 immunoglobulin, and although ipilimumab blocks interactions between CTLA-4 and CD80 or CD86, its mechanism of action is believed to include ADCC depletion of tumor-infiltrating regulatory T-cells that express high levels of cell surface CTLA-4. (Mahoney et al. (2015) NATURE REVIEWS, DRUG DISCOVERY 14: 561-584.) Given that CTLA-4 is highly expressed on a subset of T-cells (regulatory T-cells) that act to negatively control T-cells activation, when an anti-CTLA-4 IgG1 antibody is administered, the number of regulatory T-cells is reduced via ADCC.

In certain embodiments, it is desirable to use immunopotentiators whose mode of action is primarily to block the inhibitory signals between the cancer cells and the T-cells without significantly depleting the T-cell populations (for example, permitting the T-cell populations to expand). To achieve this, it is desirable to use an antibody, for example, an anti-PD-1 antibody, an anti-PD-L1 antibody or an anti-PD-L2 antibody, that has or is based on a human IgG4 isotype. Human IgG4 isotype is preferred under certain circumstances because this antibody isotype invokes little or no ADCC activity compared to the human IgG1 isotype (Mahoney et al. (2015) supra). Accordingly, in certain embodiments, the immunopotentiator, e.g., the anti-PD-1 antibody, anti-PD-L1 antibody, or anti-PD-L2 antibody has or is based on a human IgG4 isotype. In certain embodiments, the immunopotentiator is an antibody not known to deplete Tregs, e.g., IgG4 antibodies directed at non-CTLA-4 checkpoints (for example, anti-PD-1 IgG4 inhibitors).

In certain embodiments, the immunpotentiator is an antibody that has or is based on a human IgG1 isotype or another isotype that elicits antibody-dependent cell-mediated cytotoxicity (ADCC) and/or complement mediated cytotoxicity (CDC). In other embodiments, the immunpotentiator is an antibody that has or is based on a human IgG4 isotype or another isotype that elicits little or no antibody-dependent cell-mediated cytotoxicity (ADCC) and/or complement mediated cytotoxicity (CDC).

Exemplary PD-1-based inhibitors are described in U.S. Pat. Nos. 8,728,474, 8,952,136, and 9,073,994, and EP Patent No. 1537878B1. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include nivolumab (OPDIVO®, Bristol-Myers Squibb), pembrolizumab (KEYTRUDA®, Merck), cemiplimab (LIBTAYO®, Regeneron/Sanofi), spartalizumab (PDR001), MEDI0680 (AMP-514), pidilizumab (CT-011), dostarlimab, sintilimab, toripalimab, camrelizumab, tislelizumab, and prolgolimab. Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include avelumab (BAVENCIO®, EMD Serono/Pfizer), atezolizumab (TECENTRIQ®, Genentech), and durvalumab Medimmune/AstraZeneca).

In certain embodiments, a subject is administered a PD-1-based inhibitor, e.g., an anti-PD-1 antibody, e.g., an anti-PD-1 antibody contemplated herein, every 1 to 5 weeks (e.g., every 1, 2, 3, 4, or 5 weeks). In certain embodiments, a subject is administered a PD-1-based inhibitor, e.g., an anti-PD-1 antibody, e.g., an anti-PD-1 antibody contemplated herein, every 2 to 4 weeks (e.g., every 2, 3, or 4 weeks).

The PD-1-based inhibitor may be designed, expressed, and purified using techniques known to those skilled in the art, for example, as described hereinabove. The anti-PD-1 antibodies may be designed, expressed, purified, formulated and administered as described in U.S. Pat. Nos. 8,728,474, 8,952,136, and 9,073,994.

Other immunopotentiators (for example, antibodies, and various small molecules) may target signaling pathways involving one or more of the following ligands: B7-H3 (found on prostrate, renal cell, non-small cell lung, pancreatic, gastric, ovarian, colorectal cells, among others); B7-H4 (found on breast, renal cell, ovarian, pancreatic, melanoma cells, among others); HHLA2 (found on breast, lung , thyroid, melanoma, pancreas, ovary, liver, bladder, colon, prostate, kidney cells, among others); galectins (found on non-small cell lung, colorectal, and gastric cells, among others); CD30 (found on Hodgkin lymphoma, large cell lymphoma cells, among others); CD70 (found on non-Hodgkin's lymphoma, renal cells, among others); ICOSL (found on glioblastoma, melanoma cells, among others); CD155 (found on kidney, prostrate, pancreatic glioblastoma cells, among others); and TIM3. Similarly, other potential immunopotentiators that can be used include, for example, a 4-1BB (CD137) agonist (e.g., the fully human IgG4 anti-CD137 antibody Urelumab/BMS-663513), a LAG3 inhibitor (e.g., the humanized IgG4 anti-LAG3 antibody LAG525, Novartis); an IDO inhibitor (e.g., the small molecule INCB024360, Incyte Corporation), a TGFβ inhibitor (e.g., the small molecule Galunisertib, Eli Lilly) and other receptor or ligands that are found on T-cells and/or tumor cells. In certain embodiments, immunopotentiators (for example, antibodies, and various small molecules) that target signaling pathways involving one or more of the foregoing ligands are amenable to pharmaceutical intervention based on agonist/antagonist interactions but not through ADCC.

It is further envisioned that the present invention can be used in combination with surgical intervention. In the case of surgical intervention, the present invention may be used preoperatively, e.g., to render an inoperable tumor subject to resection. Alternatively, the present invention may be used at the time of surgery, and/or thereafter, to treat residual or metastatic disease. For example, a resected tumor bed may be injected or perfused with a formulation comprising the immune cell and/or superantigen conjugate. The perfusion may be continued post-resection, for example, by leaving a catheter implanted at the site of the surgery. Periodic post-surgical treatment also is envisioned. Any combination of the invention therapy with surgery is within the scope of the invention.

Continuous administration also may be applied where appropriate, for example, where a tumor is excised and the tumor bed is treated to eliminate residual, microscopic disease. Delivery via syringe or cauterization is preferred. Such continuous perfusion may take place for a period from about 1-2 hours, to about 2-6 hours, to about 6-12 hours, to about 12-24 hours, to about 1-2 days, to about 1-2 weeks or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Exemplary cytotoxic agents that can be administered in combination with a method or composition described herein include, for example, antimicrotubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In one embodiment, the cytotoxic agent that can be administered with a method or composition described herein is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids.

### VII. Kits

In addition, the invention provides kits comprising, for example, a first container containing a superantigen conjugate and a second container containing an immune cell. Such a kit may also contain additional agents such as, for example, corticosteroid or another lipid modulator. The container means may itself be a syringe, pipette, and/or other such like apparatus, from which the formulation may be applied to a specific area of the body, injected into an animal, and/or applied and/or mixed with the other components of the kit.

The kits may comprise a suitably aliquoted superantigen conjugate and/or immune cell, and optionally, lipid and/or additional agent compositions of the present invention. The components of the kits may be packaged either in aqueous media or in lyophilized form. When the components of the kit are provided in one and/or more liquid solutions, the liquid solution is a sterile aqueous solution.

### EXAMPLES

The following Examples are merely illustrative and are not intended to limit the scope or content of the invention in any way.

### Example 1

This Example describes an in vitro study testing the anti-cancer effect of the tumor-targeted superantigen conjugate naptumomab estafenatox (NAP) in combination with CAR T-cells against the FaDu head and neck tumor cell line.

Peripheral blood mononuclear cells (PBMCs) were isolated from healthy donors. PBMCS include T cells and cells comprising a major histocompatibility complex (MHC) class II (e.g. monocytes). PBMCs were incubated for 4 days with (i) 10 μg/m1 NAP and 20 units/ml IL-2, or (ii) with antibodies against CD3 and CD28 and 20 units/ml IL-2. CD8⁺ T cells were then isolated and further modified to express a CAR that has (i) an extracellular portion including variable heavy and light domains of a monoclonal anti-Her2 antibody and a hinge, (ii) a transmembrane domain, (iii) an intracellular portion including a signaling domain derived from CD3z and a costimulatory sequence derived from 41BB, and (iv) a myc tag for detection. To express the CAR, a nucleic acid encoding the CAR was cloned into pGEM4z, enabling the production of CAR-encoding mRNA by in vitro transcription. 0.25 μg of mRNA encoding either Her2 CAR or a negative control CAR (lacking the scFV) was electroporated into CD8⁺ T cells for expression for up to 48 hours.

FaDu cancer cells expressing both the antigen targeted by the CAR (Her2) and the antigen targeted by NAP (5T4) were incubated with CD8⁺ T cells for 4 hours. The effector:target ratio (T cells:FaDu cells) was 5. Where indicated, 0.1 ng/ml NAP was added to the assay. At the end of the treatment the culture supernatant was removed, including suspended T cells, and the viability of the cancer cells was tested with a CCK-8 kit (Cell Counting Kit-8, Sigma Aldrich) according to the manufacturer's protocol. The viability of the control group (no T cells) was normalized to 100%. Viability of the cancer cells (%)=(OD value of treatment group/OD value of control group)×100.

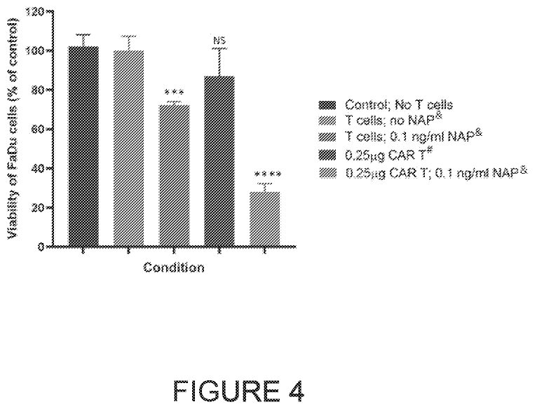

As shown in FIG. 4, Her2 CAR T cells alone (grown in the presence of CD3 and CD28) had no significant effect on the viability of FaDu cancer cells. Although the inclusion of NAP in the assay with T cells (grown in the presence of NAP) reduced the viability of tumor cells by 30% relative to the control (p=0.0007), the combination of CART cells (grown in the presence of NAP) and 0.1 μg/m1 NAP had the strongest effect, resulting in a 75% reduction in cancer cell viability (p<0.0001 vs. all test groups). These results demonstrate that administration of CAR T-cells in combination with the tumor-targeted superantigen NAP can result in an enhanced anti-cancer effect that is greater than the additive effect of each agent when administered alone.

### Example 2

This Example describes a study testing the effect of stimulation with NAP on CAR T cell potency.

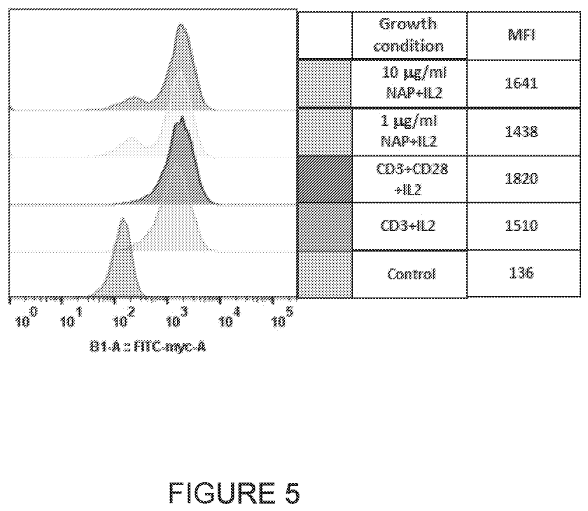

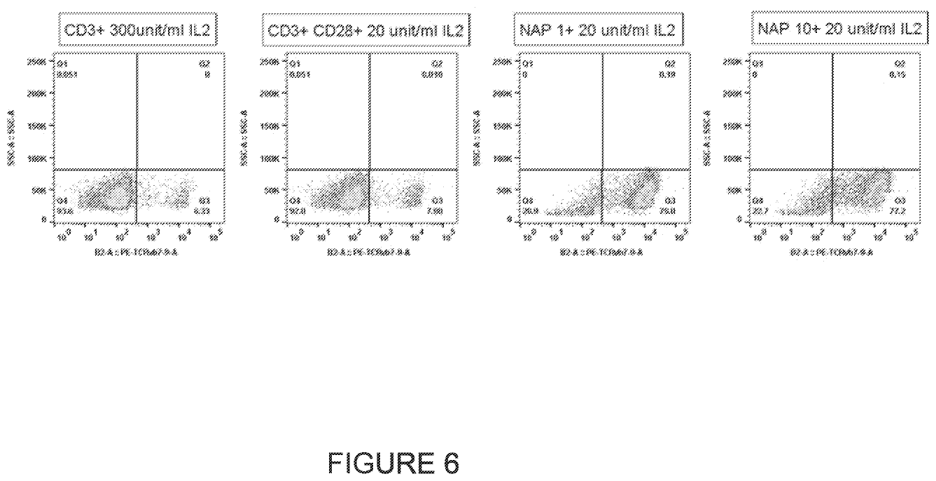

Peripheral blood mononuclear cells (PBMCs) were isolated from healthy donors. PBMCS include T cells and cells comprising a major histocompatibility complex (MHC) class II (e.g. monocytes). PBMCs were incubated with either (i) NAP (1 or 10 μg/ml) and IL-2 (20 units/ml), (ii) antibodies against CD3 and CD28 and IL-2 (20 units/ml), or (iii) an antibody against CD3 and a high dose of IL-2 (300 units/ml). Following 4 days of stimulation, CD8⁺ T-cells were isolated and rested overnight and then induced to express CAR constructs by electroporation with 1 μg of Her2 CAR mRNA as described in Example 2. On the day of the study, expression of CAR constructs was quantified by flow cytometry and was found to be similar across all activation methods (FIG. 5). TRBV7-9 expression was measured by FACS using a multimer of phycoerythrin (PE)-labeled NAP. Results showed that the percentage of TRBV7-9 CD8⁺ T cells was enriched 10-fold following NAP activation relative to the CD3/CD28 stimulations (FIG. 6).

To assess the potency of the CAR T-cells, Her2-expressing FaDu cancer cells were incubated for 4 hours with the activated Her2 CAR T-cells. NAP was not added in this assay. The effector:target ratio (T cells:tumor cells) was 5:1. At the end of the treatment, the viability of the FaDu cancer cells was determined with a CCK8 kit as described in Example 2.

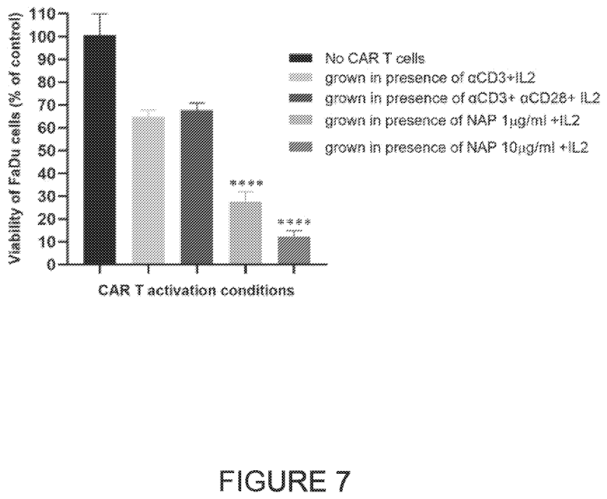

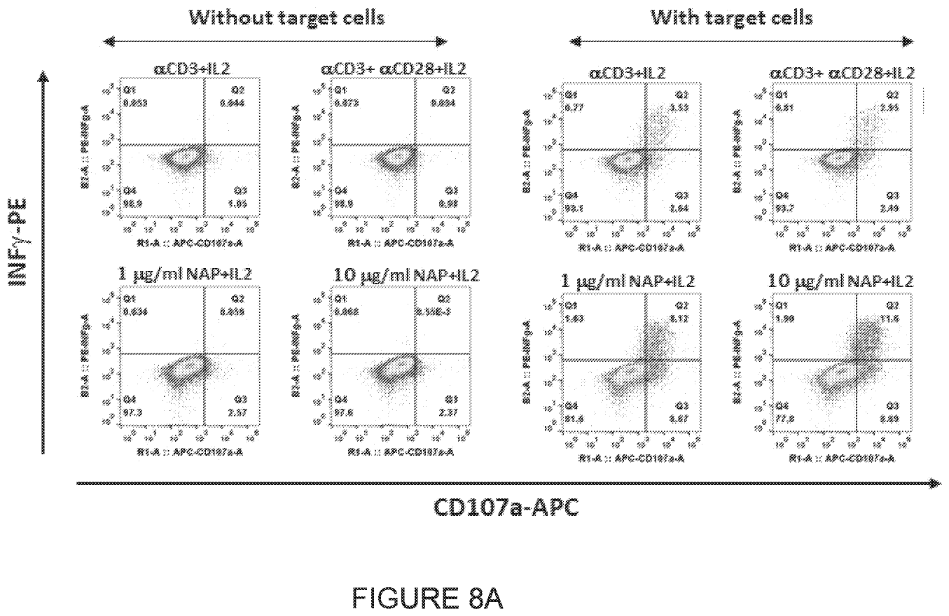

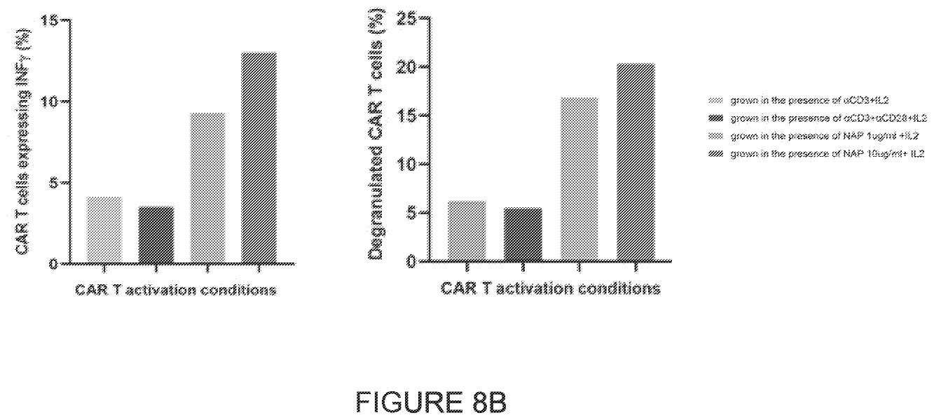

Although stimulation with NAP had no effect on CAR expression, NAP-stimulation significantly enhanced the potency of CAR T cells against FaDu cancer cells. The CD3/CD28-stimulated CAR T cells reduced cancer cell viability by about 35%, whereas the NAP-stimulated CAR T cells reduced cancer cell viability by more than 70% (p<0.0001; FIG. 7). Furthermore, a larger percentage of NAP-stimulated CAR T cells than CD3/CD28-stimulated CAR T cells expressed INFγ and the degranulation marker CD107a, which are indicators of increased T-cell activity (FIG. 8). Surprisingly, even though NAP was not present in the experimental conditions tested, prior stimulation with NAP increased CAR T cell activity.

Taken together, these results demonstrate that NAP activation significantly enhanced CAR T-cell potency and indicate that NAP-stimulation may be an improvement over standard methods including CD3/CD28-induced in vitro activation and expansion of T cells (e.g., CAR T-cells) prior to administration to patients.

### Example 3

This Example describes an in vitro study comparing the anti-cancer effect of CAR T cells in combination with either NAP or unconjugated Staphylococcal enterotoxin superantigen (SEA) against the FaDu head and neck tumor cell line.

Peripheral blood mononuclear cells (PBMCs) were isolated from healthy donors. PBMCS include T cells and cells comprising a major histocompatibility complex (MHC) class II (e.g. monocytes). PBMCs were incubated with either (i) NAP (10 μg/m1) and IL-2 (20 units/ml), (ii) SEA (10 ng/ml) and IL-2 (20 units/ml), or (iii) antibodies against CD3 and CD28 and IL-2 (20 units/ml). Following 4 days of stimulation, CD8⁺ T-cells were isolated, rested overnight and then induced to express CAR constructs by electroporation with 0.167 μg of Her2 CAR mRNA as described in Examples 1 and 2.

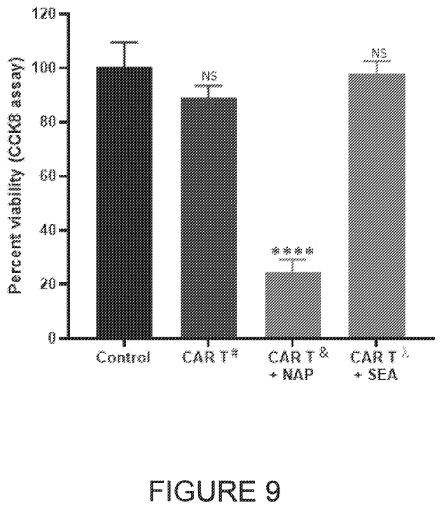

FaDu cancer cells expressing both the antigen targeted by the CAR (Her2) and the antigen targeted by NAP (5T4) were incubated with CD8⁺ T cells for 4 hours. The effector:target ratio (T cells:FaDu cells) was 5. Where indicated, 0.01 ng/ml NAP or 0.01 ng/ml SEA was added to the assay. At the end of the treatment, the viability of the FaDu cancer cells was determined with a CCK8 kit as described in Example 1. The viability of the control group (no T cells) was normalized to 100%. Results are shown in FIG. 9.

The combination of SEA and CAR T cells (grown in the presence of SEA) was ineffective against the FaDu cells. CAR T cells grown in the presence of antibodies against CD3 and CD28 were likewise ineffective. In contrast, the combination of NAP and CAR T cells (grown in the presence of NAP) reduced FaDu cell viability by 76.2% (p<0.0001; FIG. 9). These results demonstrate that the combination of CAR T cells and the superantigen conjugate NAP has a significant anti-cancer effect relative to the combination of CAR T cells and the unconjugated superantigen SEA.

### Incorporation by Reference

The entire disclosure of each of the patent and scientific documents referred to herein is incorporated by reference for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method of treating cancer in a subject in need thereof, the method comprising administering to the subject: (i) an effective amount of a superantigen conjugate comprising a superantigen covalently linked to a targeting moiety that binds a first cancer antigen expressed by cancerous cells within the subject; and (ii) an effective amount of an immune cell comprising an exogenous nucleotide sequence encoding a chimeric antigen receptor (CAR) that binds a second cancer antigen expressed by cancerous cells within the subject.

2. The method of claim 2, wherein the superantigen comprises Staphylococcal enterotoxin A or an immunologically reactive variant and/or fragment thereof.

3. The method of any one of claims 1-3, wherein the superantigen comprises the amino acid sequence of SEQ ID NO: 3, or an immunologically reactive variant and/or fragment thereof.

4. The method of any one of claims 1-3, wherein the targeting moiety is an antibody.

5. The method of claim 4, wherein the antibody is an anti-5T4 antibody.

6. The method of claim 5, wherein the anti-5T4 antibody comprises a Fab fragment that binds a 5T4 cancer antigen.

7. The method of claim 6, wherein the anti-5T4 antibody comprises a heavy chain comprising amino acid residues 1-458 of SEQ ID NO: 8 and a light chain comprising amino acid residues 1-214 of SEQ ID NO: 9.

8. The method of any one of claims 1-7, wherein the superantigen conjugate comprises a first protein chain comprising SEQ ID NO: 8 and a second protein chain comprising SEQ ID NO: 9.

9. The method of any one of claims 1-8, wherein the immune cell is selected from a T-cell, a natural killer cell (NK), and a natural killer T-cell (NKT).

10. The method of claim 9, wherein the immune cell is a T-cell.

11. The method of claim 10, wherein the T-cell comprises a T-cell receptor comprising TRBV7-9.

12. The method of claim 11, wherein the first and/or second cancer antigen is selected from 5T4, mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD47, CD49f, CD56, CD74, CD123, CD133, CD138, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-a and β (FRa and β), Ganglioside G2 (GD2), Ganglioside G3 (GD3), an Epidermal Growth Factor Receptor (EGFR), Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Ra2), K-light chain, kinase insert domain receptor (KDR), Lewis A (CA19.9), Lewis Y (LeY), LI cell adhesion molecule (LICAM), melanoma-associated antigen 1 (melanoma antigen family A1, MAGE-A1), Mucin 16 (MUC-16), Mucin 1 (MUC-1), KG2D ligands, cancer-testis antigen NY-ESO-1, tumor-associated glycoprotein 72 (TAG-72), vascular endothelial growth factor R2 (VEGF- R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-IH, HERK-V, IL-1 IRa, Latent Membrane Protein 1 (LMP1), Neural cell-adhesion molecule (N-CAM/CD56), programmed cell death receptor ligand 1 (PD-L1), B Cell Maturation Antigen (BCMA), and Trail Receptor (TRAIL R).

13. The method of claim 12, wherein the first and/or second cancer antigen is selected from 5T4, EpCAM, HER2, EGFRViii, and IL13Rα2.

14. The method of claim 13, wherein the first cancer antigen is 5T4.

15. The method of any one of claims 1-14, wherein the superantigen conjugate and the immune cell are administered separately or in combination.

16. The method of claim 15, wherein the superantigen conjugate and the immune cell are administered at the same time.

17. The method of claim 15, wherein the superantigen conjugate and the immune cell are administered at different times.

18. The method of any one of claims 1-17, wherein the method further comprises administering to the subject a PD-1 based inhibitor.

19. The method of claim 18, wherein the PD-1 based inhibitor is a PD-1 or PD-L1 inhibitor.

20. The method of claim 19, wherein the PD-1 inhibitor is an anti-PD-1 antibody.

21. The method of claim 20, wherein the anti-PD-1 antibody is selected from nivolumab pembrolizumab, and cemiplimab.

22. The method of claim 19, wherein the PD-L1 inhibitor is an anti-PD-L1 antibody.

23. The method of claim 22, wherein the anti-PD-L1 antibody is selected from atezolizumab, avelumab, and durvalumab.

24. The method of any one of claims 1-23, wherein the subject is a human subject.

25. A pharmaceutical composition comprising: (i) a superantigen conjugate comprising a superantigen covalently linked to a targeting moiety that binds a first cancer antigen expressed by cancerous cells within the subject; (ii) an immune cell comprising an exogenous nucleotide sequence encoding a chimeric antigen receptor (CAR) that binds a second cancer antigen expressed by cancerous cells within the subject; and (iii) a pharmaceutically acceptable carrier or diluent.

26. A method of treating cancer in a subject in need thereof, the method comprising administering to the subject an effective amount of the pharmaceutical composition of claim 25.

27. A method of expanding T-cells comprising a T-cell receptor comprising TRBV7-9, the method comprising contacting the T-cells with (i) a superantigen comprising Staphylococcal enterotoxin A or an immunologically reactive variant and/or fragment thereof, and (ii) a cell comprising a major histocompatibility complex (MHC) class II.

28. A method of producing a T-cell for use in the treatment of a subject, the method comprising contacting T-cells with (i) a superantigen comprising Staphylococcal enterotoxin A or an immunologically reactive variant and/or fragment thereof, and (ii) a cell comprising a major histocompatibility complex (MHC) class II.

29. A method of producing a chimeric antigen receptor (CAR) T-cell, the method comprising:
a) contacting T-cells with (i) a superantigen comprising Staphylococcal enterotoxin A or an immunologically reactive variant and/or fragment thereof, and (ii) a cell comprising a major histocompatibility complex (MHC) class II; and
b) modifying the T-cells to comprise an exogenous nucleotide sequence encoding a chimeric antigen receptor (CAR).

30. A method of producing a chimeric antigen receptor (CAR) T-cell, the method comprising:
a) modifying T-cells to comprise an exogenous nucleotide sequence encoding a chimeric antigen receptor (CAR); and
b) contacting the T-cells with (i) a superantigen comprising Staphylococcal enterotoxin A or an immunologically reactive variant and/or fragment thereof, and (ii) a cell comprising a major histocompatibility complex (MHC) class II.

31. A method of producing a chimeric antigen receptor (CAR) T-cell, the method comprising modifying T-cells to comprise an exogenous nucleotide sequence encoding a chimeric antigen receptor (CAR), wherein the T-cells have been contacted with (i) a superantigen comprising Staphylococcal enterotoxin A or an immunologically reactive variant and/or fragment thereof, and (ii) a cell comprising a major histocompatibility complex (MHC) class II.

32. A method of producing a chimeric antigen receptor (CAR) T-cell, the method comprising contacting T-cells with (i) a superantigen comprising Staphylococcal enterotoxin A or an immunologically reactive variant and/or fragment thereof, and (ii) a cell comprising a major histocompatibility complex (MHC) class II, wherein the T-cells have been modified to comprise an exogenous nucleotide sequence encoding a chimeric antigen receptor (CAR).

33. The method of any one of claims 27-32, wherein the superantigen comprises the amino acid sequence of SEQ ID NO: 3, or an immunologically reactive variant and/or fragment thereof.

34. The method of any one of claims 27-33, wherein the cell comprising an MHC class II is an antigen presenting cell (APC).

35. A T-cell prepared by the method of any one of claim 27, 28, 33, or 34.

36. A CAR T-cell prepared by the method of any one of claims 29-34.

37. A method of treating cancer in a subject in need thereof, the method comprising administering to the subject: (i) an effective amount of a superantigen conjugate comprising a superantigen covalently linked to a targeting moiety that binds a first cancer antigen expressed by cancerous cells within the subject; and (ii) an effective amount of the T-cell of claim 35 or the CAR T-cell of claim 36.

38. A method of treating cancer in a subject in need thereof, the method comprising administering to the subject an effective amount of the T-cell of claim 35 or the CAR T-cell of claim 36.

39. The method of claim 38, wherein the method does not comprise administering to the subject an effective amount of a superantigen conjugate comprising a superantigen covalently linked to a targeting moiety that binds a first cancer antigen expressed by cancerous cells within the subject.

40. A pharmaceutical composition comprising T-cells, wherein at least 10% of the T-cells comprise a T-cell receptor comprising TRBV7-9.

41. The pharmaceutical composition of claim 40, wherein at least 20% of the T-cells comprise a T-cell receptor comprising TRBV7-9.

42. The pharmaceutical composition of claim 41, wherein at least 30% of the T-cells comprise a T-cell receptor comprising TRBV7-9.

43. The pharmaceutical composition of claim 42. wherein at least 40% of the T-cells comprise a T-cell receptor comprising TRBV7-9.

44. A method of treating cancer in a subject in need thereof, the method comprising administering to the subject: (i) an effective amount of a superantigen conjugate comprising a superantigen covalently linked to a targeting moiety that binds a first cancer antigen expressed by cancerous cells within the subject; and (ii) an effective amount of the pharmaceutical composition of any one of claims 40-43.

45. A method of treating cancer in a subject in need thereof, the method comprising administering to the subject an effective amount of the pharmaceutical composition of any one of claims 40-43.

46. A T-cell modified to have increased expression of TRBV7-9 relative to a T-cell that has not been modified.

47. The T-cell of claim 46, wherein the T-cell comprises an exogenous nucleotide sequence encoding TRBV7-9.

48. The T-cell of claim 47, wherein the T-cell further comprises an exogenous nucleotide sequence encoding a chimeric antigen receptor (CAR).

49. A method of treating cancer in a subject in need thereof, the method comprising administering to the subject: (i) an effective amount of a superantigen conjugate comprising a superantigen covalently linked to a targeting moiety that binds a first cancer antigen expressed by cancerous cells within the subject; and (ii) an effective amount of the T-cell of any one of claims 46-48.

50. The method of any one of claims 1-24, 26, 37-39, 44, 45, and 49, wherein the cancer is selected from a cancer expressing 5T4, mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD47, CD49f, CD56, CD74, CD123, CD133, CD138, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-a and β (FRa and β), Ganglioside G2 (GD2), Ganglioside G3 (GD3), an Epidermal Growth Factor Receptor (EGFR), Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Ra2), K-light chain, kinase insert domain receptor (KDR), Lewis A (CA19.9), Lewis Y (LeY), LI cell adhesion molecule (LICAM), melanoma-associated antigen 1 (melanoma antigen family A1, MAGE-A1), Mucin 16 (MUC-16), Mucin 1 (MUC-1), KG2D ligands, cancer-testis antigen NY-ESO-1, tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-IH, HERK-V, IL-1 IRa, Latent Membrane Protein 1 (LMP1), Neural cell-adhesion molecule (N-CAM/CD56), programmed cell death receptor ligand 1 (PD-L1), B Cell Maturation Antigen (BCMA), and Trail Receptor (TRAIL R).

51. The method of claim 50, wherein the cancer is selected from a cancer expressing 5T4, EpCAM, HER2, EGFRViii, and IL13Rα2.

52. The method of claim 51, wherein the cancer is a 5T4-expressing cancer.

53. The method of any one of claims 1-24, 26, 37, 42, and 46-49, wherein the cancer comprises a solid tumor.

54. The method of any one of claims 1-24, 26, 37-39, 44, 45, and 49-53, wherein the cancer is selected from breast cancer, bladder cancer, cervical cancer, colon cancer, colorectal cancer, endometrial cancer, gastric cancer, head and neck cancer, liver cancer, melanoma, mesothelioma, non-small cell lung cancer, ovarian cancer, pancreatic cancer, prostate cancer, renal cell cancer, and skin cancer.


SEQUENCE LISTING (12 sequences)

SEQ ID NO: 1 - protein, 233 aa, Staphylococcus sp
SEKSEEINEKDLRKKSELQRNALSNLRQIYYYNEKAITENKESDDQFLENTLLFKGFFTGHPWYNDLLVDLGSKDATNKYKGKKVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNKTINSENLHIDLYLYTT

SEQ ID NO: 2 - protein, 233 aa, Staphylococcus sp
SEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTS

SEQ ID NO: 3 - protein, 233 aa, Artificial Sequence
  Mutated Protein
SEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 4 - protein, 233 aa, Artificial Sequence
  Mutated Protein
SEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIAIYLYTS

SEQ ID NO: 5 - protein, 679 aa, Artificial Sequence
  Conjugated Protein
  MISC_FEATURE (460)..(679): Light Chain
QVQLQQPGAELVRPGASVKLSCKASGYTFTNYWINWVKQRPGQGLEWIGNIYPSYIYTNYNQEFKDKVTLTVDESSSTAYMQLSSPTSEDSAVYYCTRSPYGYDEYGLDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTSDIVMTQSPSSLTVTAGEKVTMNCKSSQSLLNSRNQKNYLTWYQQKPGQPPKLLIYWASTRESGVPDRFTGSGSGTDFTLTISSVQAEDLAVYYCQNDYVYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 6 - protein, 672 aa, Artificial Sequence
  Mutated and Conjugated Protein
  MISC_FEATURE (459)..(672): Light Chain
EVQLQQSGPDLVKPGASVKISCKASGYSFTGYYMHWVKQSHGKSLEWIGRINPNNGVTLYNQKFKDKAILTVDKSSTTAYMELRSLTSEDSAVYYCARSTMITNYVMDYWGQVTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIAIYLYTSSIVMTQTPTSLLVSAGDRVTITCKASQSVSNDVAWYQQKPGQSPKLLISYTSSRYAGVPDRFSGSGSGTDFTLTISSVQAEDLAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 7 - protein, 672 aa, Artificial Sequence
  Mutated and Conjugated Protein
  MISC_FEATURE (459)..(672): Light Chain
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTTSIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 8 - protein, 458 aa, Artificial Sequence
  Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 9 - protein, 214 aa, Artificial Sequence
  Synthetic polypeptide
SIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 10 - protein, 233 aa, Artificial Sequence
  Mutated Protein
SEKSEEINEKDLRKKSELQGTALGNLKQIYYYNEKAITENKESDDQFLENTLLFKGFFTGHPWYNDLLVDLGSKDATNKYKGKKVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNKTINSENLHIALYLYTT

SEQ ID NO: 11 - protein, 115 aa, Homo sapiens
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSL

SEQ ID NO: 12 - DNA, 473 bp, Homo sapiens
atgggcaccagcctcctctgctggatggccctgtgtctcctgggggcaggtgagtcctcagaacaccaagcaatctcattgtgtctgtgtatgtctgtgtgtgtgtgcgtgtgtgtgtgtgtgtgtgtgatgactacaattgttttcctcctgttcccaacttgtatctccacagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttagc